(19) United States
(12) Patent Application Publication
(10) Pub. No.: US 2021/0154369 A1
(43) Pub. Date: May 27, 2021

(54) DEVICE AND PROCESS FOR PRODUCING FIBER PRODUCTS AND FIBER PRODUCTS PRODUCED THEREBY

(71) Applicant: LifeNet Health, Virginia Beach, VA (US)

(72) Inventors: Barton D. Gaskins, Virginia Beach, VA (US); Dennis L. Phelps, Virginia Beach, VA (US); Daniel B. Osborne, Virginia Beach, VA (US); Louis E. Ford, Virginia Beach, VA (US); Lloyd Wolfinbarger, JR., Norfolk, VA (US); Xiaofei Qin, Virginia Beach, VA (US)

(73) Assignee: LifeNet Health, Virginia Beach, VA (US)

(21) Appl. No.: 17/164,616

(22) Filed: Feb. 1, 2021

Related U.S. Application Data
(63) Continuation-in-part of application No. 16/179,173, filed on Nov. 2, 2018.
Application No. 16/179,173 is a continuation of application No. 15/494,001, filed on Apr. 21, 2017, now Pat. No. 10,905,801.
Application No. 15/494,001 is a continuation of application No. 12/692,879, filed on Jan. 25, 2010, now abandoned.
Application No. 12/692,879 is a division of application No. 10/606,208, filed on Jun. 26, 2003, now Pat. No. 7,744,597.
Application No. 10/606,208 is a continuation-in-part of application No. 16/059,430, filed on Aug. 9, 2018.
Application No. 16/059,430 is a continuation of application No. 14/730,458, filed on Jun. 4, 2015, now abandoned.
Application No. 14/730,458 is a continuation of application No. 11/518,566, filed on Sep. 11, 2006, now Pat. No. 9,080,141.
Application No. 11/518,566 is a division of application No. 10/835,529, filed on Apr. 30, 2004, now Pat. No. 7,494,811.
(60) Provisional application No. 60/466,772, filed on May 1, 2003.

(51) Int. Cl. A61L 27/38; A61B 17/16; A61F 2/28; A61F 2/46; A61L 27/36

(52) U.S. Cl. CPC A61L 27/3821; A61B 17/1635; A61F 2/28; A61F 2/4644; A61L 2430/02; A61L 27/365; A61L 27/3691; A61F 2002/2817; A61F 2002/4649; A61L 27/3608

(57) ABSTRACT

The present invention is directed to a fiber, preferably bone fiber, having a textured surface, which acts as an effective binding substrate for bone-forming cells and for the induction or promotion of new bone growth by bone-forming cells, which bind to the fiber. Methods of using the bone fibers to induce or promote new bone growth and bone material compositions comprising the bone fibers are also described. The invention further relates to a substrate cutter device and cutter, which are effective in producing substrate fibers, such as bone fibers. The present invention is also directed to a device for the growth of new bone or bone-like tissue under in vitro cell culture conditions.

### CROSS-REFERENCE TO RELATED APPLICATIONS

This application is a Continuation-in-Part of U.S. application Ser. No. 16/179,173, filed Nov. 2, 2018; which is a Continuation of U.S. application Ser. No. 15/494,001, filed Apr. 21, 2017, allowed; which is a Continuation of U.S. application Ser. No. 12/692,879, filed on Jan. 25, 2010, abandoned; which is a Divisional of U.S. application Ser. No. 10/606,208, filed on Jun. 26, 2003, now U.S. Pat. No. 7,744,597, issued Jun. 29, 2010; this application is also a Continuation-in-Part of U.S. application Ser. No. 16/059,430, filed Aug. 9, 2018; which is a Continuation of U.S. application Ser. No. 14/730,458, filed Jun. 4, 2015, abandoned; which is a Continuation of U.S. application Ser. No. 11/518,566, filed Sep. 11, 2006, now U.S. Pat. No. 9,080,141, issued Jul. 14, 2015; which is a Divisional of U.S. application Ser. No. 10/835,529, filed Apr. 30, 2004, now U.S. Pat. No. 7,494,811, issued Feb. 24, 2009, which claims benefit of U.S. Provisional Application No. 60/466,772, filed May 1, 2003, the contents of each of which are all hereby incorporated herein by reference in their entireties.

### FIELD OF THE INVENTION

This invention relates to a cutting device for cutting a substrate, processes for the production of substrate fibers, and the substrate fibers produced. Suitable substrates include but are not limited to bone tissue, including allogenic and xenogenic cortical bone. The fibers are cut from a substrate using the device, such that an individual fiber produced has a length that is typically greater than 10 to 200 times its width and thickness. The invention further relates to compositions including bone fibers and other agents, including, for example, bioactive agents, including stem cells, which bind to the bone fibers and are induced to form new bone.

This invention also relates to the formation of a tissue-engineered material using in vitro cell culture, in a bioreactor system(s), in the presence of biomaterials suitable for the induction of new bone formation. This invention further relates to the use of specific forms of reactors to cause the formation of a shaped material suitable to specific clinical applications. For example, the formation of a mandible-shaped reactor for in vitro growth of a shaped bone graft substitute for the use in repair of fractured jaws is within the scope of the present invention. This invention further relates to a bone forming tissue that will remodel into load-bearing bone when implanted in the surgical repair of bone defects.

### BACKGROUND OF THE INVENTION

Ground demineralized cortical and cancellous bone have been widely used in the induction of new bone formation for the treatment of a variety of clinical pathologies. Typically, the bone materials are obtained from human or animal sources, ground and demineralized. Such bone has been demonstrated over the past two decades to induce new bone formation when implanted in animal models, to stimulate elevated levels of the enzyme alkaline phosphatase, and to contain extractable amounts of bioactive molecules, such as bone morphogenetic proteins (BMPs).

The ground demineralized bone matrix (DBM) has also been called demineralized bone (DMB), and demineralized freeze-dried bone allograft (DFDBA). DFDBA materials are provided for clinical use in a freeze-dried state. DBM (or DMB) can be provided for clinical use in either a freeze-dried state or as a hydrated state—usually in some form of an aqueous carrier, for example, glycerol in GRAFTON™ (GRAFTON™ is a registered trademark of Osteotech, Inc., Shrewsbury, N.J.), pluronic polymer in DYNAGRAFT™ (DYNAGRAFT™ is a registered trademark of GenSci Regeneration Technologies, Inc., Irvine, Calif.), and collagen in OPTIFORM™ (OPTIFORM™ is a registered trademark of Regeneration Technologies, Inc., Alachua, Fla.). These various commercially available demineralized bone products primarily contain demineralized cortical ground bone distributed for clinical applications. The use of carriers with demineralized bone particles are more acceptable to clinicians because such particles acquire a static charge in the dry state making them difficult to dispense into containers and following rehydration, the clinician typically has difficulties in getting the bone particles to remain at the implant site and in a compacted state wherein they are presumed to be most osteoinductive. DBM is considered to be osteoinductive if it induces the formation of new bone, for example, at the site of clinical application. By adding carriers to the DBM, the biomaterials become easier to aliquot into containers and tend to remain tightly aggregated at the implant site making them easier to handle.

The osteoinductive nature of DBM arises from the interaction between bone-forming cells and the DBM. Such interaction takes place at both a molecular and physical level. At the molecular level, attachment of the bone-forming cells to the DBM involves the presence of “receptors” on the surface of the plasma membrane of mammalian cells that bind to “ligands” present on the surface of the biomaterial. An example of this type of attachment or binding is illustrated in the role of RGD-containing amino acid sequences in the attachment of mammalian cells to a wide variety of molecules present within matrices of tissues. The RGD amino acid sequence refers to the amino acids arginine (R), glycine (G), and aspartic acid (D). Holland, et al. (Biomaterials. 1996. 17(22):2147-56) described the research on a synthetic peptide, gly-arg-gly-asp-ser-pro-lys (GRGDSPK) (which includes the cell-adhesive region of fibronectin, and arg-gly-asp (RGD) peptide sequence covalently bound to a dialdehyde starch (DAS) coating on a polymer surface. The authors concluded that the GRGDSPK/DAS-coated surface could be substituted for an adhesive-protein coated surface in the culture of anchorage-dependent cells.

On the other hand, binding at the physical level in the context of surface patterning has been described, for example, in Goodman, et al. (Biomaterials. 1996. 17(21):2087-95). Goodman et al. described clinical and experimental investigations on manufactured surface topographies that have significant effects on cell adhesion and tissue integration stating that micro- and nano-scale mechanical stresses generated by cell-matrix adhesion have significant effects on cellular phenotypic behavior. Details of surface patterning effects on cell attachment and proliferation were described by Schmidt and Recum (Biomaterials. 1992. 13(15):1059-69) measuring macrophage responses to microtextured silicone. Schmidt and Recum measured the effects of seven different silicone surface textures on macrophage spreading and metabolic activity in vitro. Variables of the textured arrays important to cell spreading and metabolic activity included size, spacing between, depth, density, and orientation of the individual surface events and the roughness of the surfaces. It was found that pattern dimensions of about 5 microns textures were associated with small cells, whereas a smooth (untextured) surface was associated with large cells. The authors put forth a hypothesis that included a possible mechanism of how a micrometer-sized surface texture could modify cell function.

There are thus several issues pertinent to the ability of implanted bone compositions to induce the formation of bone. These issues include providing an environment suitable for the infiltration of cells, a confined environment that restricts the diffusion of synthesized matrix-forming molecules (for example, collagens, proteoglycans, and hyaluronins), promotes cell attachment to DMBs, and includes the presence of bioactive molecules (for example BMPs). Additionally, the method for making bone fibers for these bone implanted compositions in an efficient and consistent manner is addressed by the present invention.

Demineralized bone matrix (DBM) is widely used in the repair of pathologies associated with skeletal defects and periodontal diseases. This material is typically produced from cortical bone of long-bones (chiefly those bones found in the legs and arms of human cadaveric donors) by cutting the shafts of these long-bones into small chunks (1-4 mm) using methods well-known in the field. The resulting pieces and chunks of bone are subsequently cleaned and grinded into a finer bone powder. The resulting bone powder is typically in the about 125 to 1000 micron particle size ranges. The bone powder may be demineralized by exposure to dilute (normally 0.4 to 0.6 N) hydrochloric acid, organic acids, calcium chelating agents, etc. as is known in the art. For example, U.S. Pat. Nos. 5,275,954; 5,531,791; 5,556,379; 5,797,871; 5,820,581; 6,189,537; and 6,305,379 describe methods of demineralizing bone material and are hereby incorporated by reference in their entirety. This ground demineralized bone matrix material has been called demineralized freeze-dried bone allograft (DFDBA), demineralized bone allograft (DBA), demineralized bone matrix (DBM), and demineralized bone (DMB) and is currently produced by a number of for profit and not-for-profit companies for use in orthopaedic, spinal fusion, and periodontal applications.

The use of DBM in the formation of new bone has been assessed using in vivo (usually a mouse or rat implant system), in vitro (cell culture or extraction and quantitation of bone forming molecules reportedly present in bone), and in situ (where the formation of new bone in patients has been assessed during clinical applications) applications. Methods of assessing this new bone formation and the effects of the demineralization process on new bone formation by DBM are described in Zhang et al., “A quantitative assessment of osteoinductivity of human demineralized bone matrix,” J. Periodontol. 68:1076-1084 (1997) and Zhang et al., “Effects of the demineralization process on the osteoinductivity of demineralized bone matrix,” J. Periodontol. 68:1085-1092 (1997). An in vitro assessment of the ability of DBM to induce cells towards an osteoblastic phenotype has also been described (Wolfinbarger and Zheng, “An in vitro bioassay to assess biological activity in demineralized bone,” In Vitro Cell Bio. Anim. 29A:914-916 (1993)).

DBM is assumed to form new bone when implanted in animal models via an endochondral pathway. The implanted DBM is presumed to cause mesenchymal stem cells (typically undifferentiated fibroblasts) to migrate towards the implanted biomaterial(s). This induced chemotaxis results in cells infiltrating the implanted DBM biomaterial(s) where they are induced to undergo phenotypic changes from a fibroblastic cell phenotype to a chondrocyte phenotype and eventually to an osteoblast cell phenotype. These induced phenotypic changes have been reported to be due to the action(s) of one or more small molecular weight proteins falling in the TGF-β family commonly referred to as bone morphogenetic proteins (BMPs). As the change in cell phenotypes occurs, the proliferative potential of the cells declines. For example, the population doubling times increases from approximately 12 hours to approximately 40 hours. As a result, the cells synthesize and secrete collagens and other matrix-forming proteins/glycoproteins laying down a cartilagenous matrix and finally an osteoid-like matrix, which if left implanted in the animal long enough, can be shown to mineralize. This process is analogous to the formation of new bone. If the implanted materials lack the cell-inducing protein factors, only providing an environment suitable for cellular infiltration and cellular proliferation and differentiation, the implanted materials are deemed to be osteoconductive. If the implanted materials possess the cell inducing protein factors and provide an environment suitable for cellular infiltration and cellular proliferation and differentiation, the implanted materials are deemed to be osteoinductive. If the implanted materials already contain cells suitable for new bone formation, such as autogenously transplanted bone, the materials are deemed to be osteogenic.

### SUMMARY OF THE INVENTION

The present invention is directed to a fiber, preferably bone fiber, having a textured surface, which acts as an effective binding substrate for bone-forming cells and for the induction or promotion of new bone growth by bone-forming cells, which bind to the fiber. The bone fiber of the present invention may be demineralized or mineralized, or may be used in a composition comprising a combination of demineralized and mineralized bone fibers and bone particles. The bone fibers of the invention may be made from any type of bone, such as allogenic or xenogenic bone. Preferably, the bone fiber is made from cortical bone or cancellous bone, more preferably cortical bone. The bone fiber may be of any length. Preferably, the bone fiber has an average length of from about 1 mm to about 100 mm, an average width of from about 0.5 mm to about 2.5 mm, and an average thickness of from about 0.2 mm to about 1.4 mm. The fiber may then be processed according to known processes. In a preferred embodiment, the bone is freeze-dried.

The present invention further is directed to bone material compositions comprising the bone fibers of the present invention. In a preferred embodiment of this aspect of the invention, the bone material composition comprises a bone fiber and bone-forming cells, wherein the bone fiber has a textured surface, which acts as an effective binding substrate for bone-forming cells, and wherein the composition induces or promotes new bone formation from the bone-forming cells bound to the bone fiber. Preferably, the bone-forming cells are selected from stem cells, connective tissue progenitor cells, fibroblast cells, periosteal cells, chondrocytes, osteocytes, pre-osteoblasts, and osteoblasts. Most preferably, the bone-forming cells are stem cells. The bone fibers used in the bone material composition may be any type of bone, including allogenic or xenogenic bone. Preferably, the bone fibers are comprised of cortical or cancellous bone, more preferably comprised of cortical bone. In addition, the composition may further comprise cancellous bone. The composition may further comprise both demineralized and non-demineralized bone fibers or bone particles. The bone material composition may further comprise an agent effective to initiate or promote the induction of bone growth.

Yet another aspect of the invention is a method for inducing or promoting bone growth. This method comprises providing a bone fiber according to the present invention, contacting the bone fiber to bone-forming cells, which adhere to the textured surface of the bone fiber, and wherein the binding induces or promotes new bone growth from the bone-forming cells. The method may further comprise contacting the bone fibers and bone-forming cells with an agent effective to initiate or promote the induction of the new bone growth. Suitable agents to induce or promote bone growth include bone morphogenic proteins, angiogenic factors, growth and differentiation factors, mitogenic factors, and osteogenic/chondrogenic factors. Preferably, the bone fiber used in the method is demineralized. Preferred bone-forming cells include stem cells, connective tissue progenitor cells, fibroblast cells, periosteal cells, chondrocytes, osteocytes, pre-osteoblasts, and osteoblasts. Preferably, the bone-forming cells are stem cells. Moreover, the bone-forming cells may be contacted to the bone fibers via a biological fluid. Preferably biological fluids include plasma, bone marrow, blood, or blood products.

According to another aspect of the present invention a cutter is provided for producing substrate fibers. The cutter preferably includes a leading edge designed to make initial contact with the substrate and a trailing edge. The trailing edge preferably is configured such that it is raised above the leading edge by a prescribed height. The cutter includes a cutting surface upon which a blade section is disposed. The blade section is used to cut the substrate. At least one substrate channel may be provided near the blade section in order to direct the substrate fibers away from the substrate.

According to one exemplary embodiment of the present invention, the blade section can include at least one row of teeth designed specifically for cutting the substrate. Furthermore, each tooth can be configured with at least one predetermined cutting angle to reduce stress and achieve desired substrate properties. For example, one specific implementation of the invention provides a preferred primary cutting angle ranging from 3-6. Preferably the primary cutting angle can be selected to be approximately 4′ A secondary cutting angle can also be provided. The secondary cutting angle can vary between 10-18′ but is preferably selected to be approximately 14′

According to another aspect of the invention, a substrate cutting device is provided. The substrate cutting device includes a base and a tower. The base further includes a cutter that can be moved along a predetermined cutting path. A substrate chute extends through the base in order to position the substrate in a location where it will be in contact with the cutter. The tower includes a lower surface, which contains a recess. The recess can be aligned with the substrate chute. A clamping mechanism is provided to keep the substrate in contact with the cutter during the cutting process. The substrate cutting device can further include a fiber receptacle to receive the substrate fibers after they have been cut.

According to one exemplary embodiment of the present invention, the base is mounted on a slide mechanism, which moves along the predetermined cutting path. An actuation unit, such as a pneumatic actuator, can be used to supply the force necessary for moving the slide mechanism. According to one specific implementation of the present invention, the first actuation generates a force ranging between 600 lbs-900 lbs, and preferably about 750 lbs. A second actuation unit can also be provided to control the clamping mechanism. The second actuation unit can be configured to generate a force ranging from 150 lbs-250 lbs, and preferably about 200 lbs. The present invention can also include a computer controller for controlling operation of the substrate cutting device, including the first and second actuation units. For example, the computer controller can be used to adjust the force applied by the first actuation unit and/or adjust the speed at which the slide mechanism is moving. The computer controller can also be used to adjust the force applied on the substrate during the cutting process.

According to another aspect of the present invention, a method for cutting a substrate comprises the steps: placing the substrate into a substrate cutting device; applying a predetermined force on the substrate; moving a substrate cutter along a grain direction of the substrate; cutting substrate fibers from the substrate; detecting when the substrate has reached a predetermined minimum thickness; and terminating the process if the substrate has reached the predetermined minimum thickness.

The present invention is further directed to the substrate fibers produced using the substrate cutting device of the present invention.

The present invention is also directed to a method of growing new bone or bone-like tissue under in vitro cell culture conditions comprising providing ground demineralized bone and bone-forming cells in a bioreactor under conditions sufficient to form bone or bone-like tissue suitable for transplantation by causing a flow of nutrient solutions into, through, and out of the bioreactor. The bone or bone-like tissue is formed by proliferation and/or differentiation of the bone-forming cells in the presence of the ground demineralized bone and under suitable bioreactor conditions.

The bone-forming cells are preferably selected from the group consisting of stem cells, fibroblast cells, periosteal cells, chondrocytes, osteocytes, pre-osteoblasts, and osteoblasts. The most preferred bone-forming cells are fibroblast cells and pre-osteoblasts. The bone-forming cells can be autogenic, allogenic or xenogenic with respect to the intended recipient.

In accordance with the invention, the ground demineralized bone may be in the form of particles or fibers. The particles are about 50 microns to about 4 mm, preferably about 250 microns to about 710 microns. The fibers have a width of about 0.1 mm to about 0.5 mm, a thickness of about 0.05 mm to about 0.5 mm, and a length of about 1 mm to about 500 mm. If the ground demineralized bone is freeze-dried, it should be rehydrated. The invention provides that rehydration may occur either prior to or after being added in the bioreactor.

The invention further provides that additional components may be added to the bioreactor, such as collagen or hyaluronin, which may create a viscous bone-like matrix. Additionally, growth factors, such as vascular endothelial growth factor or differentiation factors such as bone morphogenetic proteins may be added.

The nutrient solution may comprise at least one of Dulbecco's modified Eagle's medium, fetal bovine serum, L-ascorbic acid-2-phosphate, antibiotics, dexamethasone, beta-glycerolphosphate, glucose, glutamine, amino acid supplements, glutathione-ethyl ester, antioxidants, caspase inhibitors, and inorganic ions suitable for mineralization-related metabolic events.

The nutrients solution may be delivered to the ground demineralized bone and bone-forming cells by resorbable hollow fibers. The hollow fibers are also sufficient to remove metabolic waste products from the bioreactor.

In another aspect of the invention, nondemineralized bone may be added along with the demineralized ground bone. The ratio of demineralized ground bone to nondemineralized bone may be about 1:1 to about 20:1 or as necessary to control availability of biologically active agents and available volume for cell growth.

The present invention is further directed to the bone or bone-like tissue formed according to the process disclosed herein. Moreover, implants comprising the bone or bone-like tissue are within the scope of the invention.

Furthermore, a method for growing an extracellular matrix capable of forming bone when transplanted into a patient is described. The method comprises providing bone-forming cells in a bioreactor under conditions sufficient to promote the growth and differentiation of cells resulting in the formation of an extracellular matrix, wherein said conditions include the flow of nutrient solutions through the bioreactor. Preferably, ground demineralized bone is added to the bioreactor. The present invention further encompasses the extracellular matrix made by this process and a method of implanting bone into a patient in need thereof comprising transplanting the formed extracellular matrix into the patient under conditions sufficient to form bone.

In yet another aspect of the invention, a device for the growth of new bone or bone-like tissue under in vitro cell culture conditions is provided. The device comprises a bioreactor, wherein the bioreactor comprises inlet and outlet ports for the flow of nutrient solutions, sample injection ports, and an inlet port and outlet port for the bioreactor to cyclically receive negative pressure and positive pressure. The bioreactor may optionally include hollow fibers for the delivery of nutrients and removal of wastes. The bioreactor is capable of applying mechanical/electrical stimuli to the formed or forming bone.

The bioreactor may further comprise an outer nondeformable chamber and inner deformable chamber. Either of these chambers may receive or remove the nutrient solutions via the inlet and outlet ports. In addition, the sample injection port may contact either chamber in which the bioreactor will receive biomaterials. Additional ports may be available to allow the bioreactor to receive cyclical negative and positive pressure in the volume between the outer nondeformable chamber and the inner deformable chamber through the inlet and outlet ports. Endplates may be used to secure the bioreactor and provide apertures to receive the ports.

Preferably, the device comprises hollow fibers, which can be in any shape. The hollow fibers can be round and tubular, or in the form of concentric rings. The hollow fibers may be made of a resorbable or non-resorbable membrane comprising polydioxanone, polylactide, polyglactin, polyglycolic acid, polylactic acid, polyglycolic acid/trimethylene carbonate, cellulose, methylcellulose, cellulosic polymers, cellulose ester, regenerated cellulose, pluronic, collagen, elastin, or combinations thereof. The pores of hollow fibers are of a specified diameter that extend from the inside to the outside of the wall of the hollow fiber. For example, the pores may have a diameter of about 2 kiloDaltons to about 50 kiloDaltons, preferably about 5 kiloDaltons to about 25 kiloDaltons, or alternatively, about 2 kiloDaltons to about 15 kiloDaltons.

In accordance with the present invention, the device may include an inner deformable chamber comprising a deformable wall. The deformable comprising a flexible permeable barrier. The flexible permeable barrier may comprise a resorbable or non-resorbable membrane made up of polydioxanone, polylactide, polyglactin, polyglycolic acid, polylactic acid, polyglycolic acid/trimethylene carbonate, cellulose, methylcellulose, cellulosic polymers, cellulose ester, regenerated cellulose, pluronic, collagen, elastin, or a combination thereof. In addition, the inner deformable chamber may further comprise a fine mesh. Preferably, the fine mesh comprises sterilizable materials and is made up of stainless steel, titanium, plastic polymer, nylon polymer, braided collagen, silk polymer, or a combination thereof. The fine mesh may have any suitable pore size range such as, for example, between about 0.1 to about 10 mm, about 1 mm and about 5 mm. The fine mesh may be on the inner surface of the flexible permeable barrier, outer surface or both.

### BRIEF DESCRIPTION OF THE DRAWINGS

FIG. 1 illustrates fiber bone having a ribbon-like structure produced using an apparatus of the present invention.

FIGS. 2A, 2B and 2C illustrate scanning electron photomicrographs of rehydrated bone fibers at a magnification of 268×. (FIG. 2A), 37×. (FIG. 2B) and 13×. (FIG. 2C), which depict the parallel striations along the grain of the bone fibers and the serrated edges and grooves which are believed to foster attachment sites for bone-forming cells.

FIG. 3 depicts a histology slide stained with Hematoxylin and Eosin of bone fibers of the present invention when implanted intramuscularly in an athymic (nude) mouse bioassay. The arrows (.fwdarw.) indicate sites of new bone formation.

FIG. 4 photographically illustrates the bone fibers when combined with cancellous particle bone to create a composite matrix as used to bind stem cells from blood, bone marrow, or similar cellular composition.

FIG. 5 is a side elevational view of a substrate cutting device according to an exemplary embodiment of the present invention.

FIG. 6 is a top plan view of the substrate cutting device.

FIG. 7 illustrates an operations cart that can be used to support the substrate cutting device and store various components.

FIG. 8 illustrates a top portion of the base of the substrate cutting device.

FIG. 9 is a top plan view of a cutter in accordance with an exemplary embodiment of the present invention.

FIG. 10A is a side elevational view of the cutter.

FIG. 10B illustrates an exemplary configuration for the cutter teeth.

FIG. 11 is a perspective view of the base of the substrate cutting device.

FIG. 12 is a top perspective view of a bottom portion of the base.

FIG. 13 illustrates the base of the substrate cutting device with all access doors in place.

FIG. 14 illustrates an exemplary substrate.

FIG. 15 is a perspective view of an exemplary tower for use with the substrate cutting device.

FIG. 16 illustrates substrate fibers produced according to one embodiment of the present invention.

FIG. 17 illustrates alternative cutters that can be used in different embodiments of the present invention.

FIG. 18 illustrates an exemplary configuration for use with one of the alternative cutters shown in FIG. 17.

FIG. 19 is a flow chart showing the steps performed when cutting substrates.

FIGS. 20A and 20B are histology slides stained with H&E of human bone fibers of the present invention (FIG. 20A) and human particle bone used as a control (FIG. 20B) implanted intramuscularly in an athymic (nude) mouse bioassay as set forth in Example 3 and, after 28 days, explanted and fixed in buffered formalin. The arrows (.fwdarw.) indicate sites of new bone formation.

FIG. 21 illustrates a broad overview of a suitable hollow fiber bioreactor system to be used in the in vitro growth of tissue suitable to the formation of bone and bone forming tissue formed thereby.

FIG. 22 illustrates the nutrient delivery and waste removal via hollow fibers of a suitable bioreactor within the scope of the present invention.

FIG. 23 illustrates “bone plug” formation in a bioreactor filled with DBM and cells.

FIG. 24 depicts a cross section of a hollow fiber bioreactor within the scope of the present invention, which assists in the calculation of the number of hollow fibers for one bioreactor.

FIG. 25 depicts another suitable hollow fiber bioreactor within the scope of the present having an inner deformable chamber.

FIG. 26 depicts femoral head formation in a hollow fiber bioreactor made according to the method of the present invention.

FIGS. 27A and 27B depict representative “bone plugs” generated in the hollow fiber bioreactor of the present invention. The dashed lines are intended for illustration purposes only. The histological analysis of these representative “bone plugs” was further depicted in FIGS. 31A to 34B. FIG. 27A depicts a “bone plug” generated from a 4 week incubation of DBM and human fibroblasts in the bioreactor. FIG. 27B depicts two “bone plugs” generated from a 4 week incubation of DBM and human fibroblasts in the bioreactor.

FIGS. 28A-28D illustrate representative “bone plugs” generated in the bioreactor that are subsequently freeze-dried. The shapes of the “bone plugs” reflect the shape of the deformable inner vessel of the bioreactor. FIGS. 28A and 28C depict freeze-dried “bone plugs” with rippled surfaces generated from a 4 week incubation of DBM and human fibroblasts in the bioreactor. FIGS. 28B and 28D depict freeze-dried “bone plugs” with smooth surfaces generated from a 4 week incubation of DBM and human fibroblast in the bioreactor.

FIG. 29 illustrates the time course of the osteocalcin levels (ng/tube) for different cell seeding densities (0.5, 1.0, 2.0, and 5.0 million fibroblast cells per 100 mg of DBM) over an incubation period of 7 weeks.

FIG. 30 illustrates the osteocalcin levels (ng/ng DNA) for various cell seeding densities (0.5, 1.0, 2.0, and 5.0 million fibroblast cells per 100 mg of DBM) on the 2nd, 3rd, 4th, 5th, and 6th week of incubation.

FIGS. 31A, 31B, and 31C illustrate the histological analysis of a “bone plug” generated in a bioreactor according to the method of the present invention at 200× magnification. The “bone plug” generated in bioreactor was embedded and sectioned. The sections were stained with the Alizarin Red (FIG. 31A), H&E (FIG. 31B), and Masson's Trichrome (FIG. 31C) methods. The Alizarin Red staining revealed the calcium deposition in newly formed extracellular matrix. H&E staining revealed the changes in fibroblast morphology and new extra-cellular matrix (ECM) production that appeared to be “osteoid” formation. Masson's Trichrome staining suggested that the newly formed extracellular matrix contained significant quantities of collagen.

FIGS. 32A-32C illustrate the histological analysis of a “bone plug” generated in a bioreactor according to the method of the present invention at 400× magnification. The sections were stained with the Alizarin Red (FIG. 32A), H&E (FIG. 32B), and Masson's Trichrome (FIG. 32C) methods. The Alizarin Red staining revealed the calcium deposition in newly formed extracellular matrix. H&E staining revealed the changes in fibroblast morphology and new extra-cellular matrix (ECM) production that appeared to be “osteoid” formation. Masson's Trichrome staining suggested that the newly formed extracellular matrix contained significant quantities of collagen.

FIGS. 33A-33B illustrate the H&E staining of a “bone plug” generated in a bioreactor according to the method of the present invention and FIGS. 33C-33D illustrate the H&E staining of an analogous “bone plug” generated from heterotopic implantation of DBM in a nude mouse (400× magnification). The new bone growth in a bioreactor after 4 weeks incubation was compared to the new bone growth in a nude mouse 4 weeks after DBM implantation. The changes in fibroblast morphology and new extracellular matrix production appeared on samples.

FIG. 34A illustrates the Mason's Trichrome staining of a “bone plug” generated in a bioreactor according to the method of the present invention and FIG. 34B illustrates an analogous “bone plug” generated from heterotopic implantation of DBM in a nude mouse (400× magnification). Significant amounts of new extracellular matrix were produced around cells and stained as collagen fibril for both “bone plug” generated in a bioreactor and explants from a nude mouse.

FIG. 35 depicts a graph of the alkaline phosphatase (nmol pNP/min/.mu.g) activity for “bone plugs” generated in a hollow fiber bioreactor with various cell seeding densities (0.5, 1, 5, and 10 millions human periosteal cells per 500 mg of DBM).

FIGS. 36A and 36B illustrate the H&E staining for a “bone plug” generated in a hollow fiber bioreactor (400× magnification) according to the method of the present invention.

### DETAILED DESCRIPTION OF THE INVENTION

The invention provides a bone fiber having surface properties that offer a suitable environment for the attachment of infiltrating cells, such that they can attach (normal mammalian cells are “attachment dependent” meaning they do not typically proliferate or maintain synthetic functions unless attached to a solid matrix) and synthesize bone matrix-forming molecules. Appropriate attachment surfaces can also contribute to the stimulation of cells to proliferate, differentiate, and to synthesize appropriate bone matrix-forming molecules.

The present invention is also directed to a method of making the bone fibers of the present invention involving the use of an apparatus suitable for cutting bone to produce fibers having the enhanced cell-binding surface to increase the bone-forming induction properties of demineralized bone material and to facilitate formation of a matrix suitable for perfusion, percolation, and infusion of viscous cell materials into the matrix.

Finally, the present invention is directed to an apparatus suitable for cutting a substrate. The apparatus includes a unique arrangement that allows the substrate to be cut into fibers having consistent properties for a particular application. A special cutter is used to cut the fibers along a grain direction of the substrate in order to produce substrate fibers. The apparatus includes various safety features, such as sensors to detect whether all access doors are shut prior to commencing operation. If a sensor is triggered during operation, the apparatus is immediately powered down in order to prevent an operator from being harmed. The present apparatus can also include a computer controller to control various operations.

### I. Definitions

The terms used herein are given their plain, ordinary meaning as understood by those having ordinary skill in the art, unless otherwise defined herein.

The “bioactive agents” of the present invention refer to the agents capable of initiating and inducing the differentiation and/or proliferation of bone cells and/or the induction of bone cell growth. The bioactive agents may include, for example, bone morphogenic proteins, stem cells, blood, blood elements, bone marrow and bone marrow extracts, platelets and platelet extracts, homogenates of skin and skin homogenate extracts, growth factors, selenium and transferrin, calcium salts, and CYMETRA™ (CYMETRA™ is a registered trademark of LifeCell Inc., New Jersey).

“Bone formation,” as used in the present invention, refers to the act of the bone-forming cells taking the form of bone cells, bone, cartilage, osteoids, and bone matrices.

The term, “bone material composition,” means a composition comprising the bone fibers or bone fibers plus anorganic or inorganic components mixed with the bone fibers of the present invention and bone-forming cells. Typically, this combination has physical characteristics that allow infusion of visous materials such as bone marrow and osteoinductive effect so as to allow the bone-forming cells to form into new bone cells under appropriate conditions.

The “cutting cycle” is a single forward plus backward stroke of the cutter across the substrate as disclosed herein.

A “cutting event” is the complete cutting run of a load chute of a substrate.

“Demineralization” refers to the act of removing minerals from tissues containing minerals. The demineralization may be conducted by processes known in the art.

The “fiber bone” or “bone fiber” of the present invention is the fiber made from bone by shaving or cutting along the length of the bone to provide the bone fiber its textured surface to which bone-forming cells may bind and the induction of bone growth may be initiated under appropriate conditions.

“Osteoinductive” shall mean the ability to induce or promote the formation of new bone either in vivo or in vitro. For example, the bone fibers of the present invention have been found to induce or promote the formation of new bone by bone-forming cells attached to its surface. The induction of new bone may be fostered by the presence of bioactive agents that assist in the initiation of this induction process.

The “substrate” of the present invention may be any material, i.e., non-biological or biological materials, which may be cut using the cutting device of the present invention. Where the substrate is bone, for example, the bone fibers act as a material upon which an organism such as bone-forming cells may grow or attach.

The term “bioreactor” is intended to mean a contained or enclosed system or vessel for the culture of cells, such as mammalian or vertebrate cells, by which sterility or the freedom from microbial contamination can be achieved. Nutrient solutions can be aseptically delivered into the bioreactor and waste solutions can be aseptically removed from the bioreactor.

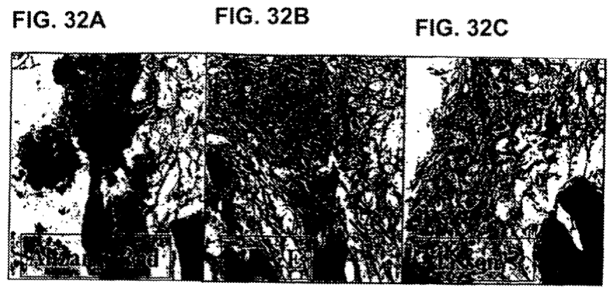

The term “newly formed bone” is intended to mean a matrix secreted by bone-forming cells. This newly formed bone is best illustrated by histological evidence of newly formed bone when demineralized bone is implanted intermuscularly in a nude mouse (or rat) bioassay system. For example, FIGS. 32A-32C depict new bone growth in a bioreactor within the scope of the present invention.

The term “bone tissue” is intended to include the organic phase or organic and inorganic phases of that tissue comprising a bone. Within the context of this invention, bone tissue can include newly formed bone, implant bone, and associated cells, bone marrow, bone marrow-like tissue, and cartilage (and cartilage-like tissues).

The term “bone-like tissue” is intended to include a matrix similar to cartilage and/or osteoid similar to that tissue found in articular cartilage, mineralized adult bone, nonmineralized fetal bone, or tissues consisting primarily of type 1, type 2 collagens, hyaluronic acid (hyluronans), proteoglycans, and non-collagenous proteins similar to those proteins found in bone and/or cartilagenous tissues. This matrix will be suitable for the growth and differentiation of chondrocytes, chondrocyte-like cells, osteocytes, osteoblasts, and/or osteoblast-like cells.

The term “transplantable bone” is intended to include a nonmineralized, partially mineralized, or fully mineralized viable construct produced, using a bioreactor, that is nonload-bearing, partially load-bearing, or fully load-bearing at the time of transplantation.

The term “implantable bone” is intended to include a nonmineralized, partially mineralized, or fully mineralized nonviable acellularized construct produced, using a bioreactor, that is nonload-bearing, partially load-bearing, or fully load-bearing at the time of implantation.

The term “strain” is intended to include forces applied to the cells and matrix contained in a bioreactor that contribute to manipulation of phenotype of the cells contained therein. As used in the present invention, strain is expected to be applied to the cells and matrix in the bioreactor through forces applied to and within the bioreactor.

The term “stress” is intended to include forces applied to the cells and matrix contained in a bioreactor that contribute to manipulation of phenotype of the cells contained therein. As used in the present invention, stress is expected to be applied to the cells and matrix in the bioreactor through forces applied to and within the bioreactor.

The term “hollow fiber” is intended to include tubular structures containing pores of defined size, shape and density for use in delivering nutrients (in solution) to cells contained within a bioreactor and for removal of waste materials (in solution) from cells contained within a bioreactor. For purposes of the present invention, hollow fibers may be constructed of a resorbable or nonresorbable material.

The term “nutrient solution” is intended to include solutions entering a bioreactor and containing those nutrient materials essential to the culture of mammalian or vertebrate cells. Nutrient solutions may also contain additives that affect specific changes in phenotype of cells under culture or to contribute to changes in the matrix structure of the forming newly formed bone, such as, mineralization.

The term “waste solution” is intended to include solutions exiting a bioreactor and containing waste byproducts of cellular metabolism. The concentrations of waste byproducts, for example ammonia, lactic acid, etc. and residual levels of nutrients such as glucose, in the waste solution can be used to assess the levels of metabolic activity of cells being cultured in a bioreactor.

It must be noted that as used herein and in the appended claims, the singular forms “a”, “and”, and “the” include plural references unless the context clearly dictates otherwise. Thus, for example, reference to “a nutrient solution” includes a plurality of such solutions and reference to “the vessel” includes reference to one or more vessels and equivalents thereof known to those skilled in the art, and so forth.

Unless defined otherwise, all technical and scientific terms used herein have the same meaning as commonly understood to one of ordinary skill in the art to which this invention belongs. Although any methods, devices, or constructs similar or equivalent to those described herein can be used in the practice or testing of the invention, the preferred methods, devices, or constructs are now described.

### II. Bone Fibers and Methods of Inducing Bone Formation

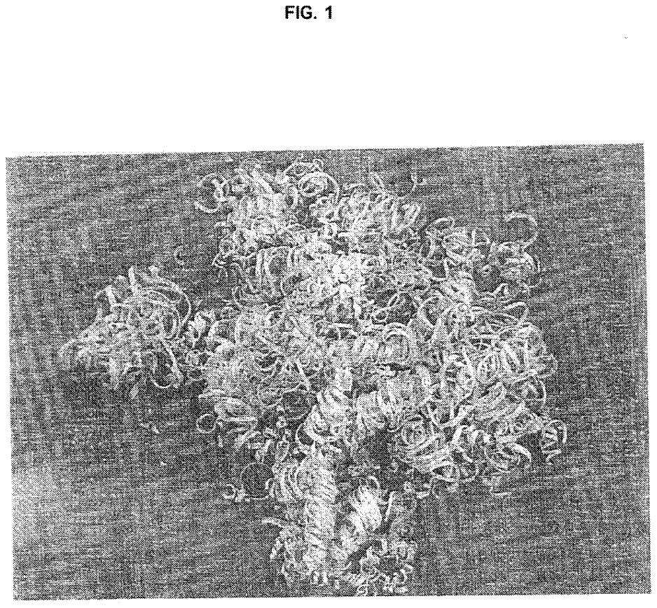

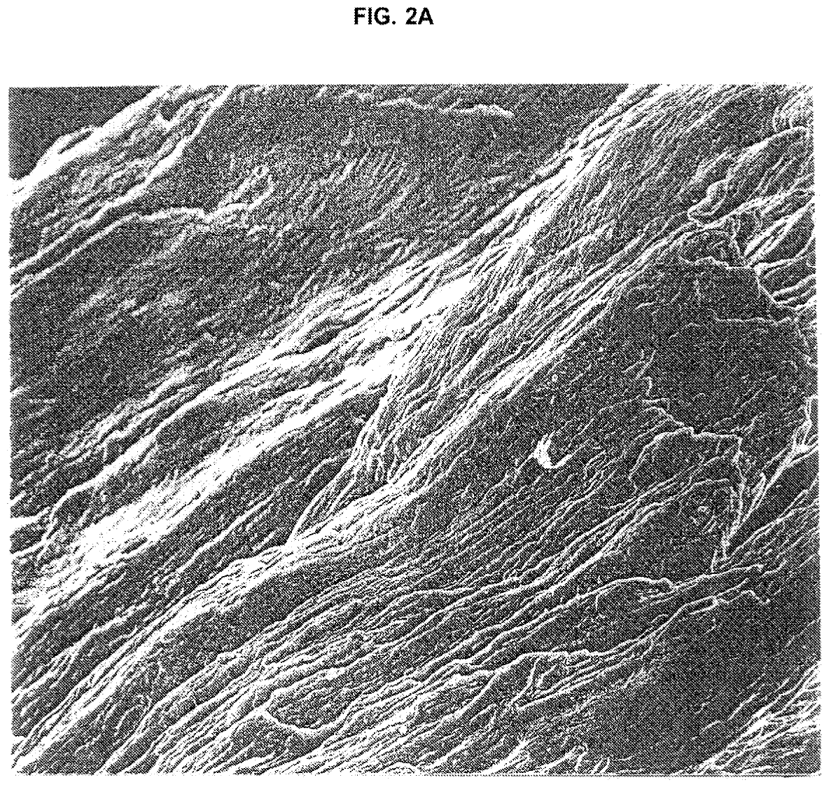

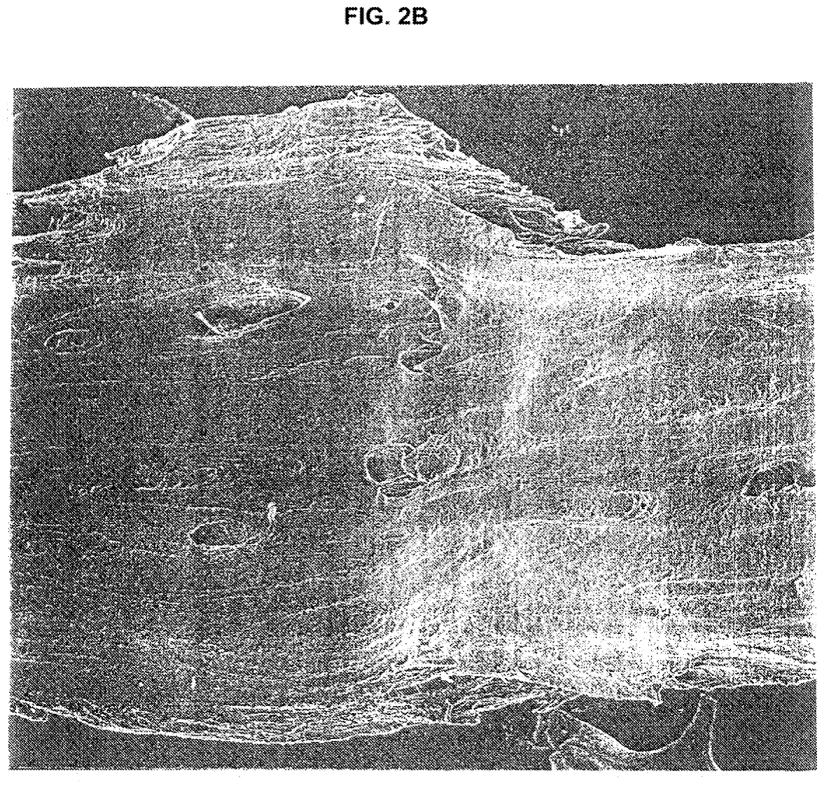

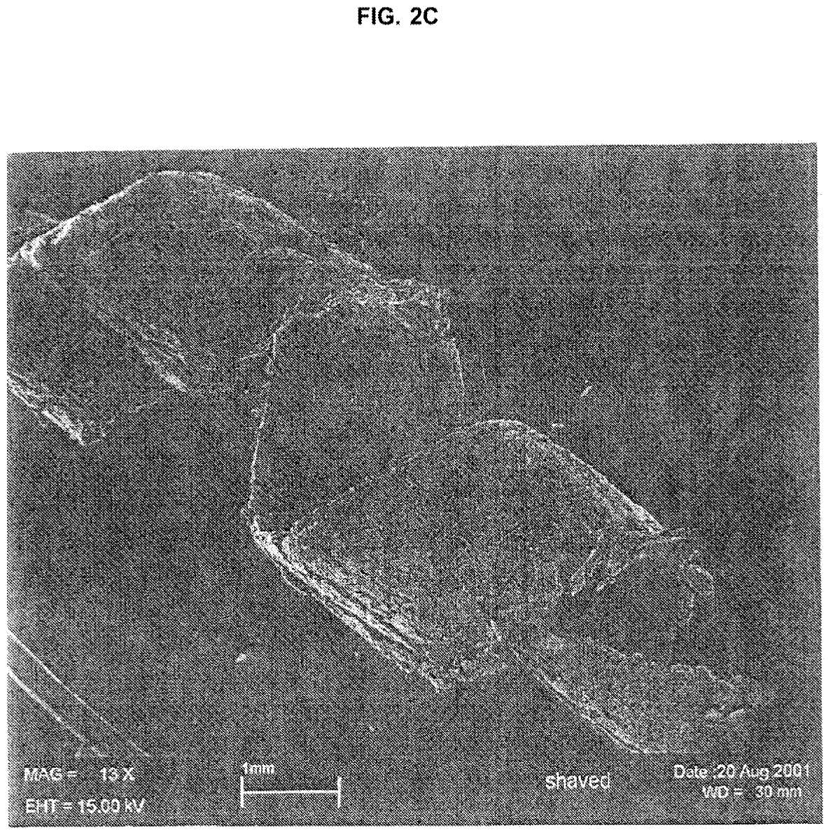

The bone fibers of the present invention have the ability to induce or promote bone formation and have properties particularly suitable as a component in bone implants. The bone fibers can be made from cortical or cancellous bone, and from any source, i.e., allograft or xenograft, by the essentially linear cutting from a bone-cutting device. The essentially linear cuttings, i.e., cuttings along the grain direction of the bone, result in bone fibers that optionally curl with the cutting process to form ribbon-like structures such as shown in FIG. 1. The fibers of the present invention preferably have a textured surface, as shown in FIGS. 2A, 2B, and 2C, having serrated edges and grooves as well as parallel striations, which provide an improved binding substrate to which bone-forming cells may attach. It is believed that this textured surface provides more available attachment sites to which bone-forming cells may adhere. Upon attachment, these cells can differentiate to form new bone and proliferate as new bone cells. Thus, the fibers of the present invention enhance the ability of bone-forming cells to bind to them so as to enhance the formation of new bone.

Fibers can be cut from any substrate that is capable of being cut using the device. Suitable substrates include non-biological materials, and biological materials. For example, suitable substrates include bone, bone tissue, plasticized bone, plasticized soft tissue, freeze-dried bone, freeze-dried soft tissue, frozen bone, frozen soft tissue, newly formed bone, implant bone, and associated cells, bone marrow, bone marrow-like tissue, cartilage, and cartilage-like tissues. Preferably, the substrate is bone tissue. Any type of bone may be used, such as allogenic and xenogenic bone. The bone tissue may be derived from any mammalian source, but is preferably human.

Production of bone fibers begins with the procurement of bone suitable to the preparation of fiber bone and includes any bone in an animal, such as bone diaphyseal shafts of long bones, for example the femur, tibia, humerus, ribs, radius, fibula. In humans, such bones are composed primarily of cortical bone tissue, but may also include cancellous bone.

The bone used to make the bone fibers may be processed in known manners prior to forming the fibers of the present invention. For example, the bone may be treated with enzymes to partially digest the organic components of the bone, such as collagenase, papain, protease, hyaluronidase, endonuclease, lipase, and/or phosphatase, or organic acids, such as acetic or citric acid. Alternatively, the bone may be partially digested by breaking or fragmenting the covalent bonds in the individual collagen molecules contained in the demineralized bone. Once the bone is cleaned of associated soft tissue, it can then be optionally cut into lengths and shapes appropriate for use in the cutting device. Alternatively the fiber bone can be cut directly from the shaft portions where the cutting blade can be attached to a manual (hand-held) cutting blade holder.

The bone tissue is used to form the bone fibers by contacting the bone tissue with an instrument capable of cutting along the length or along the grain direction of the bone tissue. The cutting instrument should be capable of cutting to provide serrated edges and grooves on the resulting bone fibers, which act as a surface-enhanced binding substrate for bone-forming cells. It has been found that bone-forming cells have an increased ability to attach to these bone fibers. While not intending to be bound by particular theory, it is believed that the edges and grooves formed on the bone fibers of the present invention provide more attachment sites to which the bone-forming cells may bind.

Cell binding to the fiber bone may be easily observed and quantitated using any number of assay methods known in the art. For example, cell populations present in any number of suspension formats, for example, bone marrow, concentrated platelets, blood, liver homogenates, etc., can be incubated with the fiber bone. The fiber bone can then be separated from the cell solution(s), gently washed to remove loosely adherent cells and other biomaterials present in the cell suspensions. The cells retained on the fiber bone can be quantitated using the traditional methyltetrazolium (MTT) assay where an insoluble chromogenic compound is formed due to the presence of metabolically viable cells (active mitochondrial enzymes) where fiber bone incubated with the suspension format lacking cells is used as a control. Alternatively, the fiber bone can be fixed with any number of fixatives, for example, formalin, and the DNA in adherent cells stained for visualization using light microscopy. The phenotypic identity, for example, fibroblasts, chondroblasts, osteoblasts, etc. can be verified using traditional enzyme assays such as alkaline phosphatase activity stains. Fibroblasts (less differentiated cells) stain only minimally for this enzyme, whereas chondrogenic and osteogenic cells stain heavily for this enzyme.

In the method of inducing new bone formation, the bone fibers of the present invention may be used in either a mineralized or demineralized state or a combination thereof. Whether mineralized or demineralized, the bone fibers have the textured surface to which the bone-forming cells may efficiently attach. In the case of demineralized bone fibers, the ribbon-like structures typically unwind into essentially linear strips of bone.

The bone fibers may be of any length, width, and thickness as deemed necessary or useful for its intended use. For example, the fibers may be the length of bone tissue from which they are being made. Alternatively, the fibers may be designed to be cut at shorter lengths to accommodate their use in particular bone implants. Bone fibers preferably have average length of from about 1 mm to about 100 mm, an average width of from about 0.5 mm to about 2.5 mm, and an average thickness of from about 0.2 mm to about 1.4 mm, more preferably having an average length of from about 20 mm to about 30 mm, an average width of from about 1.0 mm to about 2.0 mm, and an average thickness of from about 0.4 mm to about 0.8 mm. Furthermore, it is noted that the length of the fibers produced according to the invention may be substantially greater than the width and thickness of the fibers. For example, the bone fibers may have a length that is greater than about 10 to about 200 times its width and thickness, preferably about 40 to about 100 times its width and thickness. As will be described further herein, the cutting apparatus of the present invention may be modified to accommodate any desired length, width or thickness of the fibers.

For demineralization, the mineral content of the bone fibers may be removed using any known process for demineralization causing the bone fibers to be demineralized. Preferably, the bone fibers are demineralized to contain calcium at a level of from about 0.5 wt % to about 4.5 wt %, more preferably from about 1.0 wt % to about 4.0 wt %, and most preferably from about 1.5 wt % to about 3.5 wt %, for example, as disclosed in U.S. Pat. Nos. 6,189,537 and 6,305,379; and co-pending U.S. patent application Ser. Nos. 09/655,711 and 10/180,989, the disclosures of which are herein incorporated by reference in their entireties. Once demineralized, the bone fibers may optionally be combined with agents including for example, biological carriers, bioactive agents, or other agents including for example, surface active agents, preservatives including for example glycerol, and inorganic mineral compositions, either before or after further processing, such further processing including but not limited to, freeze-drying, terminal sterilization processes, and/or retaining as a hydrated fiber bone in the presence or absence of preserving agents, or combined immediately prior to implantation in a patient. Moreover, the bone fibers of the present invention may be further combined with other carriers and agents as one having ordinary skill in the art would appreciate for the use DMBs. For example, suitable biological carriers include collagen, gelatin, saccharides, fibrin, fibrinogen, alginates, hyaluronins, methylcelluloses, and biologically compatible thixotropic agents. Suitable bioactive agents include but are not limited to, bone morphogenic proteins, stem cells, blood, blood elements, bone marrow and bone marrow extracts, platelets and platelet extracts, homogenates of skin and skin homogenate extracts, growth factors, selenium and transferrin, calcium salts, and CYMETRA™.

Production of demineralized bone biomaterials and the induction of new bone by these biomaterials are described in U.S. Pat. Nos. 5,275,954, 6,189,537 and 6,305,379, of which are herein incorporated by reference in their entireties. The bone fibers of the present invention may induce or promote new bone formation by serving as a source of one or more chemoattractants that diffuse from the bone biomaterials to cause cells to migrate to the implanted bone fibers wherein cells adhere to the bone particles (normal mammalian cells are “attachment dependent,” meaning they typically require attachment to some surface in order to function metabolically) and differentiate towards a chondrocytic (cartilage forming) or osteocytic (bone forming) phenotype. In accordance with the present invention, it is believed that surface characteristics of the bone fibers of the present invention render the fibers more accessible and are a more accepting substrate to receive and bind bone-forming cells. Thus, the surface characteristics of the bone fibers may result in improved cell attachments, and consequently, act as a means for selectively attaching cartilage or bone-forming cells from a mixed population of cells, such as are present in platelet-rich plasma, blood, blood products, or bone marrow.

In accordance with the present invention, the surface patterning present on the bone fibers preferably contains parallel striations, cracks, and serrated edges and grooves to which cells may attach. This surface pattern of the bone fibers of the present invention permits a multitude of cells to bind to the bone fibers allowing less specific cells to bind and grow based on the functional properties of the fibers.

In a preferred embodiment of the present invention, the surface patterning of the bone fibers is created by the bone-cutting device of the invention as described herein. As illustrated in FIGS. 2A, 2B, and 2C, the cutting “bits” (blades) of the fiber bone-cutting device cause a micro-fractured surface with specific patterns of parallel surface striations on the cut surface of the fiber bone. Thus, in addition to the normal osteoinductive properties of demineralized bone (as described in U.S. Pat. No. 6,189,537), the bone fibers of the present invention, whether demineralized or not demineralized, present a micro-patterned surface that is not only biocompatible with bone-forming cells, but also presents a surface conducive to cellular attachment, cell spreading, and cell proliferation/differentiation (or maintenance of phenotype of an already differentiated cell). The available surface area of fiber bone produced by the present fiber bone cutting device, as compared to normal particle bone (produced by impact fragmentation), is greater, is biocompatible, and presents the surface patterning conducive to cellular attachment, proliferation, and differentiation. Due to the individual and multiple cutting bits present on a cutter present within the fiber bone cutting device, the multiplicity of patterns on the multiple fiber bone fibers produced would contribute to maximal availability of optimal surface patterning for cellular attachment. Thus, the fibers of the present invention can have variable sizes, spacing between striations, depths, density, and orientations. Preferably, the fibers are cut along the grain to obtain a greater durability of the fiber.

The “bone-forming cells” or “bone-matrix forming cells” of the present invention are those cells suitable for the induction of new bone formation when infiltrated with the bone fibers of the present invention and include those cell types suitable for differentiating into bone cells or suitable for forming a matrix similar to osteoid of forming new bone. Suitable cell types may include differentiated, partially differentiated, or undifferentiated cells. For example, cell types include, but are not limited to stem cells, connective tissue progenitor cells, fibroblast cells, periosteal cells, chondrocytes, osteocytes, pre-osteoblasts, and osteoblasts. Preferably, the stem cells are multipotent, the fibroblast cells are undifferentiated, the periosteal cells are partially differentiated, and the chondrocytes or osteocytes are differentiated.

Preferably, the bone-forming cells are stem cells. Stem cells represent a population of cells present throughout the body of mammals that are undifferentiated possessing the potential for differentiating into virtually any other, more differentiated, cell in the body. For this reason, stem cells represent a unique opportunity to repair and/or remodel damaged tissues such as broken bones, abraded cartilage, skin, etc.

Moreover, the bone-forming cells may be tissue progenitor cells. For example, U.S. Pat. Nos. 5,824,084 and 6,049,026 (and U.S. patent application 2002/0161449) describe kits and composite bone grafts contained in the kits, wherein the composite bone graft(s) are designed to contain an enriched population of connective tissue progenitor cells and a greater number of connective tissue progenitor cells per unit volume than found in the original bone marrow aspirate.

In one aspect of the invention, the bone fibers of the present invention allow for the formulation of “bone material compositions” comprising the bone fibers for use in bone implants. These bone material compositions provide increased accessibility of the bone fibers to bone-forming cells by permitting suitable voids through which viscous solutions of platelet rich plasma, bone marrow, blood or blood products may flow. For example, the bone fibers may be demineralized and compacted to form a bone material composition suitable for implantation. Because the bone fibers of the present invention are easily handled without breaking apart, the bone fibers may be molded to create an implantable composition, which retains its shape in the implant and further has appropriate spacing through which such solutions comprising bone-forming cells may pass. These bone material compositions may further have integrated therein other components, such as inorganic particles, organic particles, or more specifically non-demineralized cancellous or cortical bone chunks, which may increase the ability of such solutions to flow through the composition by providing structural spacing of the fiber bone. Under such conditions, the surface of the fiber bone fibers would be presented to the infiltrating bone marrow/platelet rich plasma preparations to promote cellular attachment, selectively concentrating the cells most appropriate to the formation of bone or cartilage when the bone material composition is then implanted into some clinical site in the body. Such ex-vivo exposure of the bone fiber biomaterials to osteogenic or chondrogenic cells would serve to concentrate cells that would normally be expected to migrate into the implanted materials through the normal chemoattractive properties of demineralized bone. Thus, this pre-implantation exposure of cells to the bone fibers should reduce the time required for the initiation of new bone formation and lessen the clinical times needed to affect a repair of the damaged site in the body, i.e. a broken bone or fusion site in an intervertebral fusion procedure for repair of cervical or lumbar complications in the spine. Other suitable components for integration into the bone material include, but are not be limited to, inorganics such as particulate calcium salts, such as calcium phosphates, calcium sulfates, and/or calcium carbonates, organics such as particulate skin, particulate cartilage, particulate tendons and ligaments, particulate dextrans, particulate alginates, and particulate resorbable and non-resorbable synthetic polymeric materials.

The bone material compositions may be formed in manners known in the art. In one embodiment of the present invention, the bone fibers of the present invention and bone-forming cells are preferably placed in a bioreactor capable of simulating the nutrient flow and waste removal present within an implant site. The flow of nutrient solutions into, through, and out of the bioreactor permit the associated ground demineralized bone and bone-forming cells to form into bone or bone-like biomaterial suitable for transplantation. In the present instance, the bioreactor use aspect of the present invention would simulate the actions of the fibers and fiber bone compositions when used clinically. The process of making bone in a bioreactor is described in Application No. 60/466,772, for example, which is herein incorporated by reference.

In another aspect of the invention, the bone fibers of the present invention have exhibited superior properties for the formation of bone implants. Bone implants may be formed using the bone fibers of the present invention based on their ability to be easily handled for molding, retaining its shape, and allowing appropriate spacing for biological solutions to pass therethrough even upon compaction. For example, the fibers may be hydrated, which renders then pliable and malleable, but capable of retaining its shape without losing durability. In fact, the fibers have been shown to retain its integrity even upon hydration, molding, and subjection to other bone implant-forming treatments. Therefore, the bone fibers of the present invention have superior properties making them ideal for the formation of bone implants.

In another aspect of the invention, the bone fibers can be used alone or in conjunction with a bone material composition and placed in a suitable container through which blood, blood products, bone marrow, or platelet rich plasma can be induced to flow through such that the cells capable of adhering to the bone material composition, specifically the fiber bone, are suitably concentrated for implantation into a site in the body wherein the formation of new bone is desired.

### III. Production of Bone Fibers

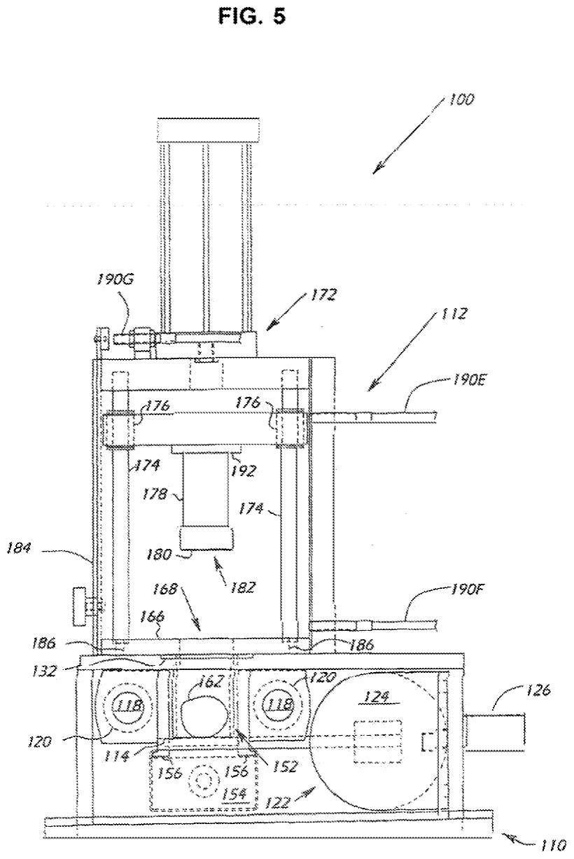

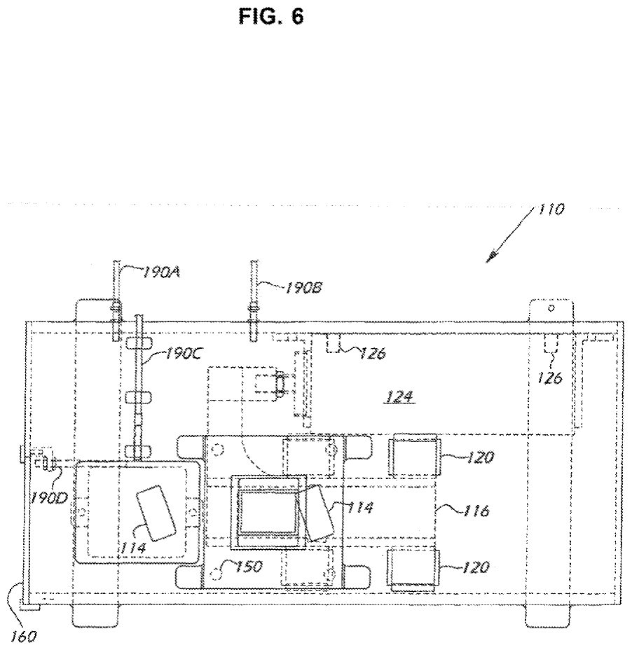

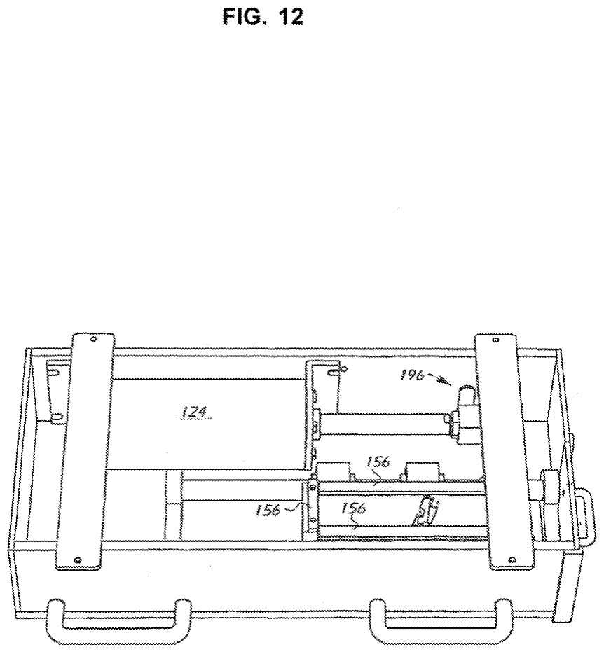

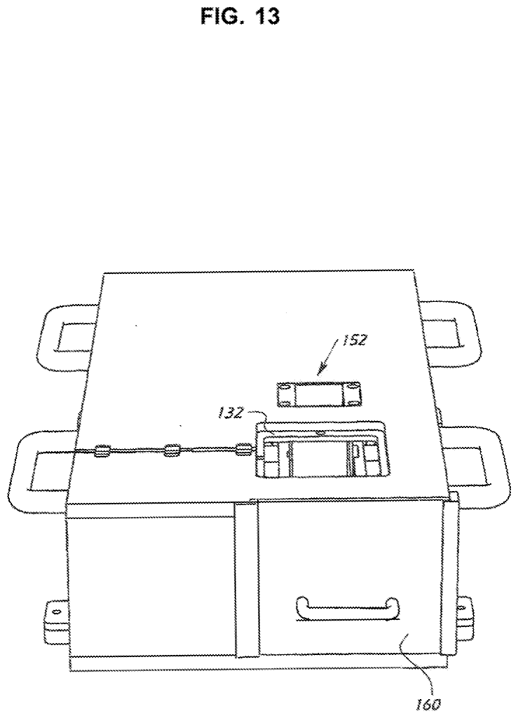

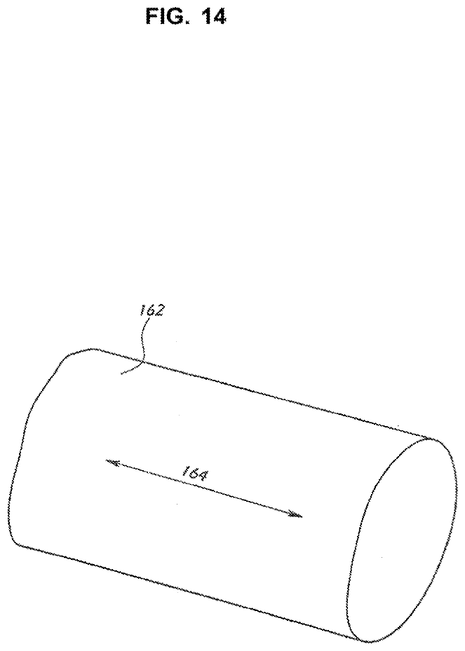

Referring to FIG. 5, a device for cutting substrates in accordance with the present invention will now be described. The substrate cutting device 100 includes a base 110, a tower 112, and a cutter 114. With continued reference to FIG. 5 and additional reference to FIG. 6, the base 110 of the substrate cutting device 100 includes a slide mechanism 116 which travels along a predetermined cutting path. Preferably, the cutting path is along with, or substantially parallel to, a grain 164 (see FIG. 14) of the substrate being cut. A pair of guide rods 118 is used to control the direction of the slide mechanism 116 during operation. A plurality of bearings 120 are also used to slidably engage the guide rods 118.

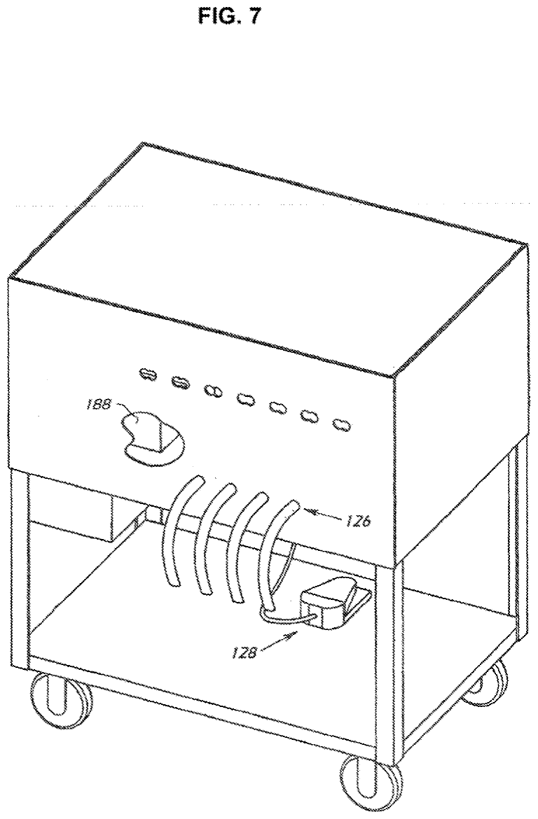

A first actuation unit 122 generates to force necessary to move the slide mechanism 116. According to the disclosed embodiment of the invention, the first actuation unit 122 is pneumatically operated. It should be noted, however, that the first actuation unit 122 can also be operated hydraulically, electrically, and/or mechanically depending on the specific requirements. As illustrated in FIGS. 5 and 6, the first actuation unit 122 includes an air cylinder 124 that receives pressurized air to generate the forces necessary for moving the slide mechanism 116. Referring additionally to FIG. 7, a plurality of pneumatic cables 126 are used to supply air to the air cylinder 124. Preferably, the air is pressurized at an external location and transferred to the substrate cutting device 100. According to such an arrangement, the pressurized air can optionally be processed in order to maintain sterile environment, when necessary. FIG. 7 also illustrates a foot pedal 128 which can be used to control the operation of the substrate cutting device 100. A computer controller 188 can also be provided to monitor and control operation of the substrate cutting device 100.

According to the disclosed embodiment of the invention, the first actuation unit 122 is configured to generate a force ranging from 600 lbs to 900 lbs. Preferably, the first actuation unit 122 generate a force ranging from 700 lbs to 800 lbs. Most preferably, the force is approximately 750 lbs. Additionally, the force can be varied during operation of the substrate cutting device 100, or it can be maintained at a constant level. For example, according to one embodiment of the invention, the computer controller 188 can be used vary the force applied by the first actuation unit 122 by reducing the amount of force applied during a return stroke and increasing the force applied during a cutting stroke.

As best illustrated in FIG. 8, the top surface of the base 110 includes a cutter access 130 which allows an operator to mount the cutter 114 within the substrate cutting device 100. The top surface of the substrate cutting device 100 also includes a substrate chute 152 designed to appropriately position a substrate 162 (see also FIG. 14) so that it may be engaged by the cutter 116. The dimensions of the substrate chute 152 can vary depending on the specific substrate and product desired. The various parts of the substrate cutting device 100 can be secured using a variety of means such as, for example, threaded fasteners 150 or any appropriate method capable of providing the strength and/or function necessary for proper operation. FIG. 8 also illustrates that the cutter 114 is rotated such that it is offset from the cutting path when mounted on the slide mechanism 116. The specific rotational offset can be selected based on a variety of factors including, but not limited to, the type of substrate, the specific arrangement of the blade sections on the cutter, and the amount of force being applied by the first actuation unit.

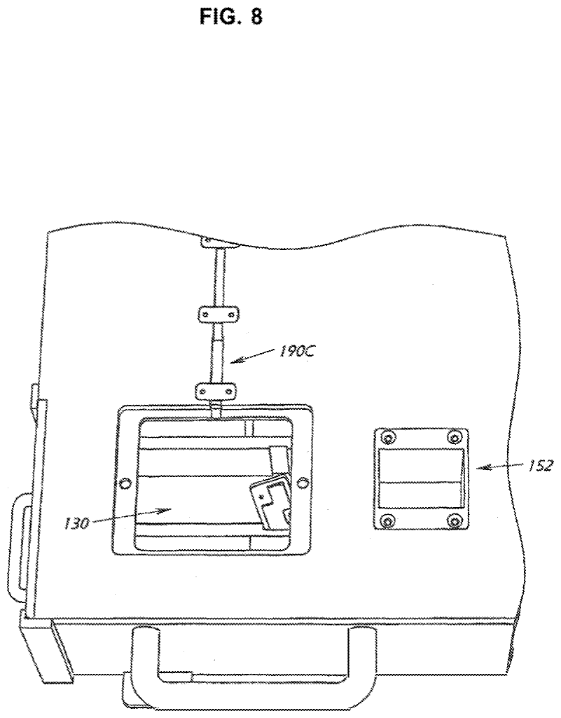

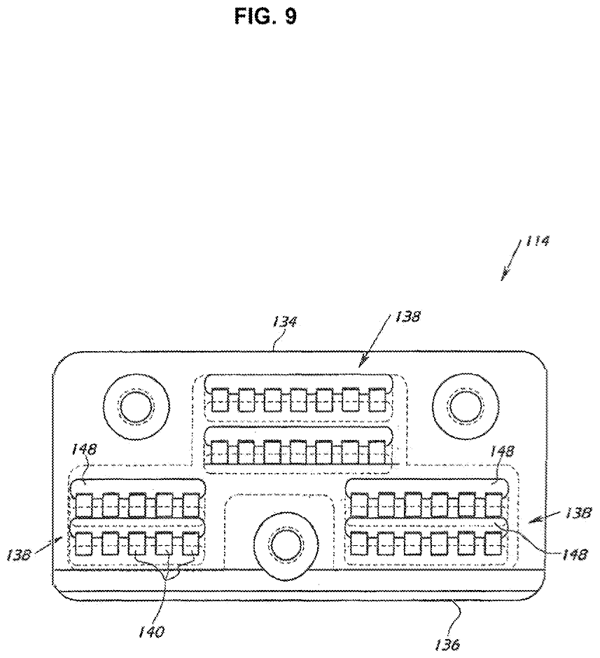

Turning to FIG. 9, the details of the cutter 114 will now be described. The cutter 114 includes a leading edge 134 and a trailing edge 136. During a cutting stroke, the leading edge 134 is the first portion of the cutter 114 to reach the substrate 162. It should be noted, however, that the leading edge 134 will not necessarily contact the substrate 162. The cutter 114 includes a plurality of blade sections 138 disposed on its surface. Each blade section 138 contains two rows of teeth 140. Depending on the specific application, desired product, and substrate, the cutter 114 can include a single blade section 138 or multiple blade sections 138 (as shown in FIG. 9). Additionally, a single row of teeth, or multiple rows of teeth may be provided.

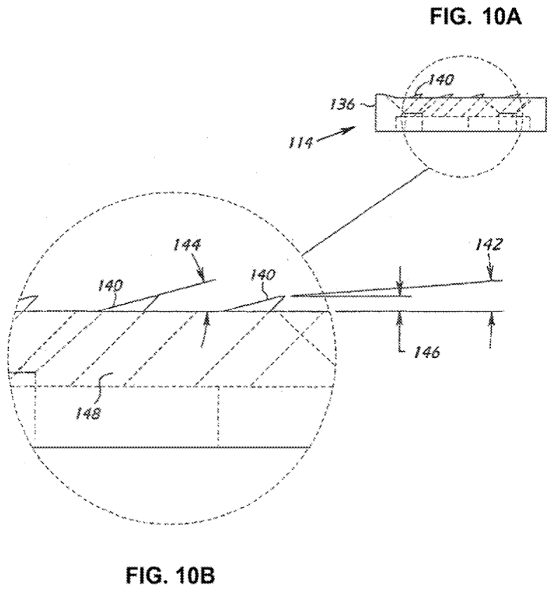

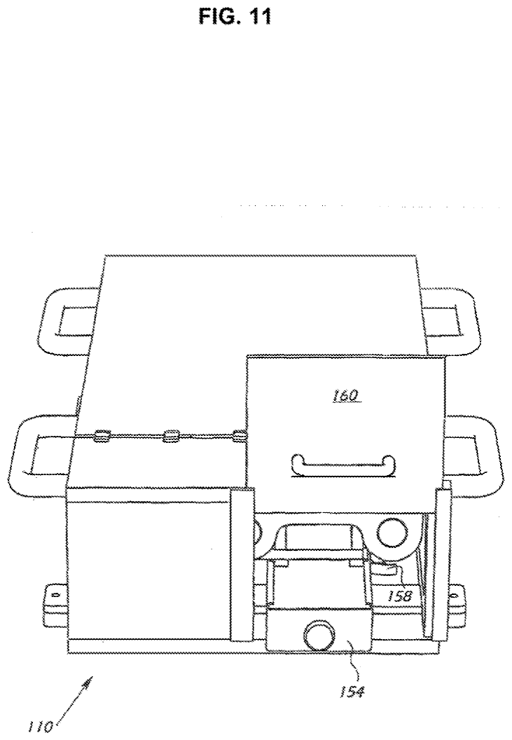

According to the disclosed embodiment of the present invention, when the cutter 114 is mounted on the slide mechanism 116, the cutter surface is substantially flush with the surface of the slide mechanism 116. Such a configuration advantageously minimizes movement of the substrate 162 during operation. Furthermore, as shown in FIG. 10A, the trailing edge 136 of the cutter 114 is raised by a predetermined amount. Preferably, this predetermined amount is approximately equal to the height of the teeth 140 in the blade section 138 in order to further minimize possible movement of the substrate 162 during operation. Referring additionally to FIG. 13, once the cutter 114 has been securely mounted to the slide mechanism 116, a cutter access door 132 is used to prevent access to the cutter 114 during operation of substrate cutting device 100.

Referring to FIGS. 10A and 10B, each tooth 140 in the blade sections 138 can include one or more cutting angles. In addition, the cutting angle can be independently selected for each individual tooth 140. More particularly, one tooth may include a single cutting angle while an adjacent tooth can include two cutting angles, and yet another adjacent tooth can contain three cutting angles. As disclosed in FIG. 10B, each tooth 140 contains a primary cutting angle 142 and a secondary cutting angle 144. The primary cutting angle 142 can be selected to be in the range of 3 to 6. Preferably, the primary cutting angle 142 is selected to be approximately 4.

The secondary cutting angle 144 can be selected in the range of 10 to 18. The secondary cutting angle 144 can also range from 12 to 16. Preferably, however, the secondary cutting angle 144 is selected to be approximately 14. FIGS. 10A and 10B also illustrate a cutting height 146 for the teeth 140. The cutting height 146 can vary depending on the specific operation and/or product desired. For example, the cutting height 146 can be used to define the thickness of fibers produced. The cutter 114 also includes a plurality of fiber channels 148 to allow passage of substrate fibers after being cut. The fiber channels 148 can be generally selected to correspond with the number of blade sections 138. More particularly, the cutter 114 is designed such that the cut substrate fibers pass directly through the fiber channel 148. Furthermore, the fiber channel 148 can be sized to assist in the production of substrate fibers having required features for a particular product. For example, by selecting an appropriate width for the fiber channel 148, the cut fibers can be prevented from curling back into the fiber channel 148 and possibly breaking prematurely. Likewise, selection of an appropriate depth for the fiber channel 148 can prevent fibers from curling into adjacent fiber channels 148.

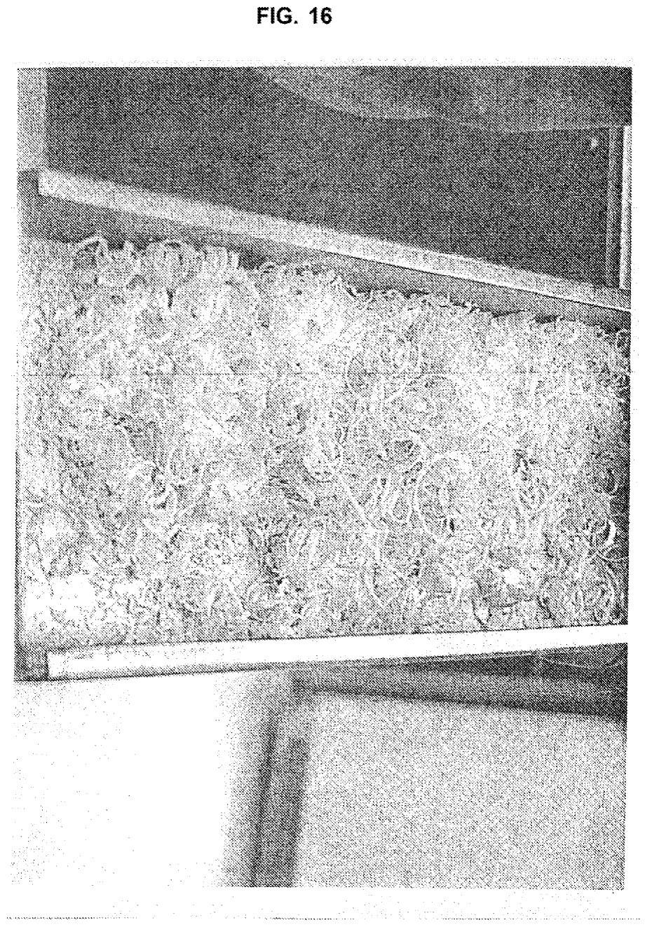

Turning now to FIGS. 11 and 12, additional features of the base 110 will be discussed. The substrate cutting device 100 can include a fiber receptacle 154 for collecting substrate fibers that have been cut. FIG. 16 illustrates a plurality of fibers that have been collected in the fiber receptacle 154. The fiber receptacle 154 is inserted into the base 110 such that it is aligned with the cutter 114 and the fiber channels 148. Accordingly, the cut fibers will fall directly into the fiber receptacle 154. A plurality of guides 156 (best seen in FIG. 12) are provided to properly align the fiber receptacle 154. A locking clip 158 can optionally be used to secure the fiber receptacle 154 in place. It should be noted, however, that various other methods and arrangements can be used to secure the fiber receptacle 154 in place. A receptacle door 160 is used cover the fiber receptacle 154 and prevent access during operation of the substrate cutting device 100. The receptacle door also includes a reflector (not shown), such as the reflector 196 on the slide mechanism 116, that allows sensor device 190d to determine whether the receptacle door 160 is closed.

Turning again to FIG. 6, the base 110 includes a plurality of sensor devices 190(a-d). The sensor devices 190 are preferably optical, but can incorporate various other detection methods as is well known. Sensor device 190a detects when the slide mechanism 116 has reached the rest (or home) position. Sensor device 190b detects when the slide mechanism 116 has completed the cutting stroke. Sensor device 190c detects the presence of the cutter access door 132. As previously indicated, sensor device 190d detects the presence of the receptacle door 160. Under normal circumstances, if sensor devices 190c and 190d return a fault, then operation of the substrate cutting device 100 is immediately halted. Additionally, sensor devices 190a and 190b can be used to monitor movement of the slide mechanism 116.

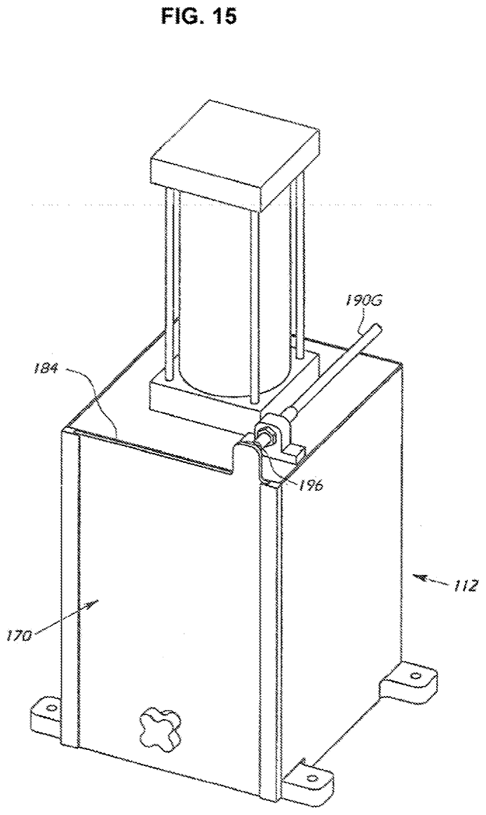

Referring to FIG. 15, with additional reference to FIG. 5, the tower 112 includes a lower surface 166 having a recess 168 therethrough. The recess 168 is positioned such that it can be aligned with the substrate chute 152. The tower 112 includes an opening 170 on a front portion thereof. The opening 170 is used to allow placement of the substrate 162 within the substrate chute 152. Once the substrate 162 is in placed in the substrate chute 152, a clamping mechanism 178 is used to keep the substrate 162 in contact with the cutter 114.

A second actuation unit 172 is used to generate the force necessary to operate the second actuation unit 172. As illustrated in the embodiment of the invention shown in FIG. 5, the second actuation unit 172 is pneumatically controlled. It should be noted, however, that hydraulic, mechanical, electrical, and other control systems can be used, so long as they are capable of supplying the force necessary to operate the clamping mechanism 178. According to the disclosed embodiment of the invention, the second actuation unit 172 is capable of generating a force ranging from 150 lbs to 250 lbs. Preferably, the second actuation unit 172 generates a force of approximately 200 lbs. Similar to the first actuation device 122, the force can be varied during operation of the substrate cutting device 100 or it can be maintained at a constant level. Additionally, the computer controller 188 can be used to monitor and/or vary the force applied by the second actuation unit 172.

As shown in FIG. 5, the clamping mechanism 178 includes a contact surface 180 that engages the substrate 162. According to a preferred embodiment of the invention, the contact surface 180 contains a plurality of grooves 182 designed to increase the friction force between the clamping mechanism 178 and substrate. The tower also includes a door 184 which prevents access during operation. One or more locating pins 186 can be used to quickly and easily align the tower 112 with the base 110. Additionally, a clamp stopper 192 can be used to prevent the clamping mechanism 178 from traveling too far and coming into contact with the cutter 114.

According to the disclosed embodiment of the invention, the tower 112 includes three sensor devices 190(e-g). Sensor device 190e detects when the clamping mechanism 178 is in the “up” (or home) position. Sensor device 190f detects when the clamping mechanism 178 is in the vicinity of the clamp stopper 192. Accordingly, sensor device 190f and the clamp stopper 192 both function to prevent accidental contact with the cutter 114. Sensor device 190g detects the presence of the door 184. If an error signal is obtained from sensor device 190g, then operation of the substrate cutting device 100 is immediately halted.

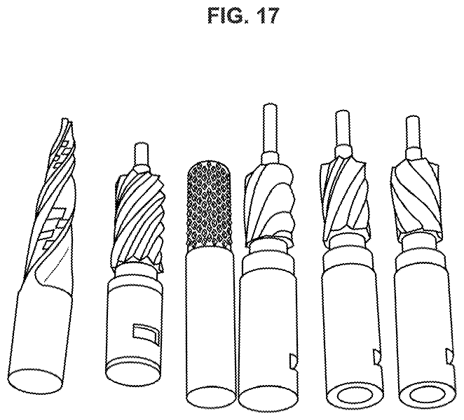

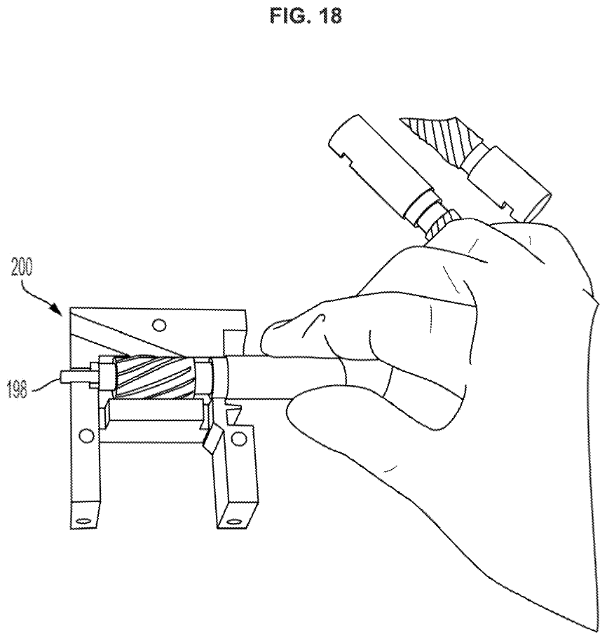

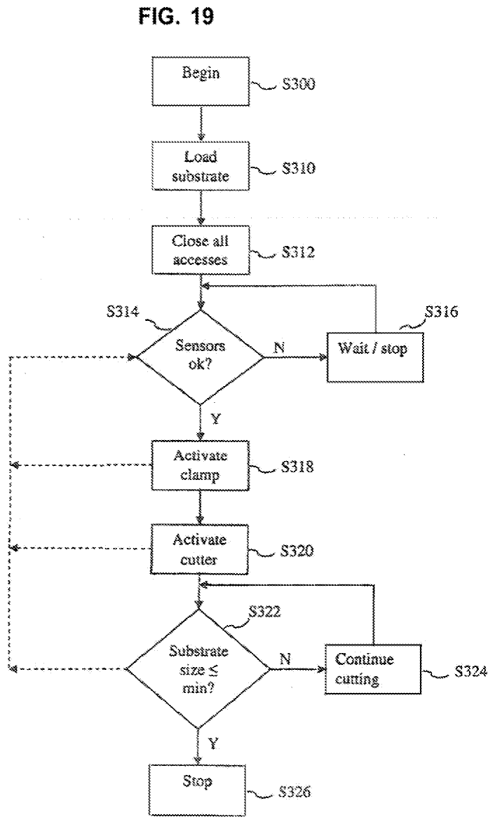

FIGS. 17 and 18 illustrates a plurality of wheel type cutters 198 (or wheel cutters) that can be used with an alternative embodiment of the present invention. The wheel cutters 198 are mounted on a base such that they may be rotated and brought into contact with the substrate. The wheel cutters 198 can be designed with various features to produce fibers having desired properties. For example, the thread depth of the wheel cutters 198 can be increased in order to produce fibers having an increased thickness. Varying the pitch of the wheel cutter 198 will effect the length and curvature of the fibers produced. As shown in FIG. 19, a substrate path 200 is used to bring the substrate in contact with the wheel cutter 198. When the pitch of the wheel cutter 198 rotates clockwise relative to the substrate, a “pulling” effect results. This requires less force on the substrate during the cutting process, and produces fibers that are short and curly. When the pitch of the wheel cutter 198 rotates counter-clockwise relative to the substrate, a greater force must be applied in order to maintain contact with the wheel cutter 198. However, the resulting fibers can be longer and will have very consistent dimensions.

FIG. 19 is a flowchart illustrating the steps performed to produce fibers in accordance with an exemplary embodiment of the present invention. The process begins at step S300. At step S310, the substrate is loaded into the substrate chute. At step S312, all of the access doors (i.e., cutter access door, receptacle door, and tower door) are closed. At step S314, the sensor devices are checked to verify that all access doors are currently closed. If any of the access doors are open, control passes to step S316. The system waits a predetermined amount of time, for example 10 seconds, and checks the sensor devices again. Alternatively, the system could continuously check the sensor devices until all access doors are closed.

Once all access doors are determined to be closed, control passes to step S318. The clamp is then activated. As previously discussed, this can be accomplished by second actuation unit applying pressure on the substrate. At step S320, the cutter is activated. At step S322, the sensor devices are checked to see if the substrate size has been reduced to a thickness, which is less than a minimum value. If the substrate thickness is greater than the minimum value, then control returns to step S322 and the cuter remains active, i.e., continues to cut the substrate. If the substrate thickness is less than or equal to the minimum value, then the system is stopped as step S326.

As illustrated by the dashed lines, the system continuously monitors the state of the sensor devices throughout the process. Thus, if any of the access doors are opened during operation of the substrate cutting device, control will pass to step S316 and the system will be immediately halted. As previously discussed, this is done, in part, to prevent injury to an operator. The system continues to operate until the either the substrate thickness reaches the minimum size, or one of the access doors is opened.

### IV. In Vitro Growth of Tissues Suitable to the Formation of Bone and Bone Forming Tissue Formed Thereby

The present invention provides a method of growing bone in vitro involving providing a biomaterial, such as ground demineralized bone, suitable for inducing cells to form an extracellular matrix and cells capable of forming bone or bone-like biomaterials, and placing the biomaterial and bone-forming cells in close association under conditions suitable for forming bone or bone-like biomaterial. In particular, the ground demineralized bone and bone-forming cells are preferably placed in a bioreactor capable of simulating the nutrient flow and waste removal present within an implant site. The flow of nutrient solutions into, through, and out of the bioreactor permit the associated ground demineralized bone and bone-forming cells to form into bone or bone-like biomaterial suitable for transplantation.

The biomaterial, ground demineralized bone, is capable of inducing selected cell types to form an extracellular matrix consistent with the osteoid materials comprising the organic phase of bone tissue when implanted in heterotopic or orthotopic sites in a living organism. Ground demineralized bone is obtained in manners known in the art and may be available in any form, including as particles or fibers. Ground demineralized freeze-dried bone particles may be used in any particle size suitable for inducing the growth of bone in a bioreactor, such as from about 50 microns to 4 mm, preferably, about 125 microns to 850 microns, and most preferably, about 250 microns to 710 microns. Ground demineralized bone fibers may be produced in known manners, such as by skiving or shaving the surface of the cortical bone to produce short fibers that easily entangle. The fibers are suitable for growing bone in a bioreactor and preferably have physical dimensions of about 0.1 mm to 0.5 mm in width, 0.05 mm to 0.5 mm in thickness, and 1 mm to 500 mm in length. The bone used to make the ground demineralized bone may be processed in known manners prior to forming the ground demineralized bone used in connection with the present invention. For example, the bone may be treated with enzymes to partially digest the organic components of the bone, such as collagenase, papain, protease, hyaluronidase, endonuclease, lipase, and/or phosphatase, or organic acids, such as acetic or citric acid. Alternatively, the bone may be partially digested by fragmenting the covalent bonds in the individual collagen molecules contained in the demineralized bone. The covalent bond breakage of the formed fragments of a collagen molecule may be in the range of about 2 to about 50, and should be sufficient to modify the resorption rate of the demineralized bone. Subsequent to forming the fibers or particles, the fibers and particles are demineralized by exposure to dilute (about 0.4 to 0.6 N) hydrochloric acid or organic acids, calcium chelating agents, etc., as one skilled in the art would appreciate. Alternatively, non-acid chelators of calcium, such as ethylene diamine tetraacetic acid (EDTA), may be used to demineralize the bone.

In addition, the weight percent residual calcium in ground demineralized bone is a factor in defining the bioavailability of bioactive molecules, such as, for example, bone morphogenetic proteins (BMPs), to the cellular population contained within the bioreactor. In fact, it has been found that the ability to extract BMPs from ground bone particles has been shown to be approximately a linear function of the extent of demineralization of the ground bone. Thus, a suitable amount of residual calcium is that amount sufficient to optimize the bioavailability of bioactive molecules, such as BMPs, to the bone-forming cells in the bioreactor. Preferably, the residual calcium is present in the range of about 0-8 weight percent, more preferably about 1-4 weight percent, and most preferably about 2 weight percent.

In accordance with the present invention, the ground demineralized freeze-dried bone particles are added aseptically to the bioreactor. They may be directly added to the bioreactor in a freeze-dried state and rehydrated in the bioreactor or rehydrated in culture medium prior to addition to the culture chamber of the bioreactor. The ground demineralized bone may be added alone or in combination with other components. Preferably, the other components do not inhibit the effect of the ground demineralized bone to induce bone formation. For example, ground nondemineralized bone may be added with ground demineralized bone. In such cases, the ground demineralized bone to nondemineralized bone may be added in any ratio, but preferably is added in a ratio of about 1:1 to about 20:1, more preferably about 8:1, and most preferably about 3:1. The ground nondemineralized bone may take any form, e.g., particles or fibers, and typically will have similar physical dimensions as the ground demineralized bone.

Particle size ranges of the ground demineralized bone particles in the bioreactor determine the “void volume” or available volume outside of the ground demineralized bone particles in which the bone-forming cells and other components may be added. It has been found that the bone particle spacing or availability of space around the ground demineralized bone particles within the bioreactor relates to the void volume and has an impact on the ability of bone-forming cells in the bioreactor to differentiate and/or proliferate. It is desired that bone-forming cells have sufficient contact to allow those cells to infiltrate the voids or space between the ground demineralized bone particles, which permits the in vitro growth of bone or bone-like tissue. Therefore, the void volume or spacing around the ground demineralized bone particles should be that which is effective in allowing for the optimal contacting and infiltration of voids by bone-forming cells between the ground demineralized bone particles.

In accordance with the present invention, the ground demineralized bone particles may be rehydrated in the bioreactor or prior to being added to the bioreactor. Preferably, the particles are rehydrated and mixed with bone-forming cells prior to addition to the bioreactor. The ground demineralized bone particle spacing will differ depending on whether or not the bone particles are rehydrated prior to addition to the bioreactor growth chamber. First, the ground demineralized bone particles may be added to the bioreactor growth chamber and subsequently rehydrated prior to adding bone-forming cells. In this approach, the ground demineralized bone particles may be added to the bioreactor growth chamber in a freeze-died state, which provides a relatively simple step and allows the particles to pack tightly filling the available space. Subsequent rehydration of these freeze-dried ground demineralized bone particles in the bioreactor will cause the bone particles to swell to a tighter state of packing due to rehydration. The bone-forming cells may then be added to the rehydrated bone matrix void volume (that volume outside of the bone particles) in the bioreactor. It has been found this tighter state of packing ground demineralized bone particles in the bioreactor is effective in more tightly packing the added bone-forming cells. While the tight packing may hinder some infiltration of the void volume present throughout the bioreactor, it has been found that the more tightly packed added cells promotes better retention of synthesized matrix molecules during the differentiation process and may be best utilized when seeding more differentiated cells into the bioreactor system.

Alternatively, the ground demineralized bone particles may be rehydrated prior to the addition to the growth chamber of the bioreactor. The bone-forming cells may then be added to the packed ground demineralized bone particles in the bioreactor or directly to the rehydrated bone particle suspension prior to its addition to the bioreactor. While rehydrating freeze-dried ground demineralized bone particles prior to addition to the growth chamber of the bioreactor has been found to increase the difficulty in adding the bone particles to the bioreactor, it has been found that directly adding the bone-forming cells to the rehydrated ground demineralized bone particle suspension results in fully dispersed bone-forming cells and ground demineralized bone particles. More uniform distribution within the growth chamber is thereby achieved and is less likely to contribute to damage to the hollow fibers present within the growth chamber.

In either case, centrifugal forces can be used to cause the rehydrated bone particles and cells to pack throughout the growth chamber with excess fluids removed from the packing port.

The “bone-forming cells” of the present invention are those cells suitable for the induction of new bone formation when infiltrated with ground demineralized bone in a bioreactor and include those cell types suitable for differentiating into bone cells or suitable for forming a matrix similar to osteoid of forming new bone. Suitable cell types include, but are not limited to stem cells, fibroblast cells, periosteal cells, chondrocytes, osteocytes, pre-osteoblasts, and osteoblasts. Preferably, the stem cells are multipotent, the fibroblast cells are undifferentiated, the periosteal cells are partially differentiated, and the chondrocytes or osteocytes are differentiated. In the case of differentiating cell types, such as fibroblasts or stem cells, these cell types may be placed in close proximity to the ground demineralized bone, which, in the bioreactor and under appropriate conditions, will cause the cells to differentiate into bone cells. In the case of cell types suitable for forming an osteoid-like matrix, such as osteoblasts or chondroblasts, such cell types may be placed in close proximity to the ground demineralized bone in the bioreactor and under appropriate conditions, will cause the cells to synthesize matrix similar to osteoid of forming new bone. The type of cells selected for in vitro bone growth is dependent upon the desired time frame for new bone formation, seeding cell densities, and nutrient medium provided.

The source of the bone-forming cells may be autogenic, allogenic, or xenogenic. The use of a potential recipient's own cells in the formation of the bone or bone-like biomaterial will result in a tissue unlikely to be rejected for some immunological reason, rendering the transplantable newly formed bone autogenous in nature. The use of allogenic cells in the formation of new bone with subsequent implantation can be achieved by decellularizing any newly formed bone or bone-like structure prior to implantation using any decellularizing technology known in the art depending on the desired characteristics of the acellular bone or bone-like structure desired for a given clinical application.

The bone-forming cells are added either to the void volume space of the packed ground demineralized bone particles or directly to the rehydrated ground demineralized bone particles prior to addition to the growth chamber of the bioreactor. The cell density of the bone-forming cells may be in the range of from about 10²-10⁸ cells per ml, preferably 10³-10⁶ cells per ml, and more preferably about 10⁴-10⁵ cells per ml. The density of bone-forming cells added depends on several factors. For example, previous cell culture work in development and validation of in vitro bioassays for assessing the osteoinductive potential of demineralized bone demonstrated the importance of cell density difference depending on the phenotypic status of the cells. (Wolfinbarger, L and Y. Zheng. 1993. An in vitro bioassay to assess biological activity of demineralized bone. In Vitro Cell Dev. Biol. Anim. 29:914.) Less differentiated cells (e.g., dermal fibroblasts), where proliferation constituted a component of the differentiation process, involved a lower seeding density in in vitro bioassays than more differentiated (periosteum derived cells, for example) cells. Presumably, cells more differentiated along the pathway leading from a “stem-like” cell to a differentiated cell phenotype proliferated less well (longer population doubling times of approximately 40 hours) than less differentiated cells (shorter population doubling times of approximately 12 hours) and could be seeded at higher cell densities when used in an in vitro bioassay. Consequently, seeding densities of cells in the bioreactor depends in part on the phenotype of the cells added to the bioreactor, the availability of biologically active materials, and the culture medium used. In addition, seeding cell density in the bioreactor depends on the ability to deliver nutrients to the cells and remove waste byproducts from the bioreactor culture chamber. For example, greater cell densities in the bioreactor require more nutrient delivery and greater waste product removal than lower cell densities.

The bioreactor can be in virtually any shape based on the shape of the bioimplant desired as a newly formed bone or structure that will form load-bearing bone when implanted clinically. The wall of the bioreactor can be deformable and contained within a nondeformable chamber such that positive and negative pressure environments can be applied between the inner wall of the nondeformable chamber and the outer wall of the deformable chamber such that the volume of the bioreactor containing the demineralized bone, cells, and matrix can be decreased or increased over time to simulate stress and strain application to the bone matrix being formed.

The demineralized bone and bone-forming cells can be preloaded into the bioreactor in the presence, or lack thereof, of a viscous matrix designed to provide attachment sites for the cells and/or to restrict diffusion of synthesized osteoid forming molecules. The viscous nature of the matrix may be obtained by the incorporation of polymers, for example, collagenous, hyaluronin, or similar resorbable or nonresorbable polymers.

Nutrients are delivered to the ground demineralized bone and bone-forming cells in the bioreactor and may impact the growth and differentiation of cells contained in the bioreactor. The nutrient solutions are selected to provide sufficient nutrition to the bone-forming cells to maintain viability, growth, and/or differentiation in the bioreactor. Those skilled in the art are capable of selecting an appropriate nutrient solution for the present invention. For example, media such as Dulbecco's modified Eagle's medium may be used and may be further supplemented with other suitable nutrients. Other suitable nutrients include fetal bovine serum, L-ascorbic acid-2-phosphate, antibiotics, cell modulators such as dexamethasone, beta-glycerolphosphate, glucose, glutamine, amino acid supplements, inhibitors (or activators) of apoptosis such as glutathione-ethyl ester, antioxidants, caspase inhibitors, and cations and anions, e.g., magnesium, manganese, calcium, phosphate, chloride, sodium, potassium, zinc, and sulfate ions, and nitrates and nitrites. The concentration of fetal calf serum must not inhibit induced cell differentiations due to diffusible agents from the demineralized bone. The remaining concentration of components in the nutrient solution should be sufficient to promote growth and/or differentiation in the bioreactor and maintain viability of the bone-forming cells and the resulting bone or bone tissue.

In accordance with the present invention, the nutrient solutions may be modified during different phases of the process. For example, during initial culture, seeded cell densities may be minimal, especially for fibroblast cell seeding cultures, and thus nutrient solutions may contain low concentrations of fetal calf serum (such as <2% vol:vol) to facilitate the role of growth and differentiation factors diffusing from the ground demineralized bone particles in modulating phenotypic changes in the added cells. Monitoring the concentration of the nutrients, such as glucose, glutamine, and amino acid supplements, via the eluent flow of medium allows for the determination of nutrient consumption permitting control of flow (delivery) of nutrients into the cell population. Moreover, waste products of metabolism, for example, ammonia and lactic acid, can be monitored via the eluent flow of medium from the bioreactor to determine the metabolic state/function of the resident cell population. Changes in cell phenotype during the culture phase can be monitored by sampling the eluent flow of medium from the bioreactor for proteins associated with specific cell phenotypes, for example, osteopontin and osteocalcin. Should it be desired, for example, other components may be added to the medium during culture to promote a desired function. For example, to induce mineralization during a specific phase of the culture period, chemical components such as β-glycerolphosphate may be added to the medium as a substrate for alkaline phosphatase and to serve as a source of phosphate to be complexed with calcium in the formation of crystalizable calcium salts such as hydroxyapatite. Alternatively, hormonal stimulation of cells can be accomplished via the addition of certain compounds such as, for example, vitamin D. The levels of oxygen tension can be controlled by oxygenation of the nutrient medium being added to the cells being cultured in the bioreactor to manipulate the metabolic state of the cells during the culture phase such that mildly hypoxic conditions can be used to manipulate chondrogenesis and/or osteogenesis. Manipulation of the ionic composition of the medium can be used to control hydrolytic enzyme degradation of demineralized bone matrix, enzyme mediated cross-linking of the formed extracellular matrix being synthesized by the resident cell population, and the osmotic balance of the nutrient solution. Induction and/or inhibition of cellular apoptosis can be controlled by the addition of inhibitors (or activators) of apoptosis such as glutathione-ethyl ester, antioxidants, and caspase inhibitors or activators. For example, use of allogenic cells may require induction of apoptosis to produce a cellular formed bone tissue. In addition, gamma irradiation treatment of the bone particles, either before or after demineralization, can be used to promote cell-mediated resorption of the demineralization bone particles facilitating new bone formation within the areas where the bone particles are resorbed.

The nutrients may be delivered in any manner suitable for the formation of bone in the bioreactor. For example, resorbable hollow fibers can be used to deliver nutrients and remove metabolic waste products during the cellular proliferations and/or differentiation process. The nutrient solutions used can be sequentially introduced into the bioreactor growth chamber as needed to induce cellular morphogenesis, growth, secretion of osteoid biomaterials, and/or to cause mineralization of the formed matrix as desired depending on the type of implantable bone material desired. The resorbable hollow-fibers used to deliver nutrients and remove wastes from the bone forming part of the bioreactor provide an opportunity to leave a series of hollow tube-like openings within the formed bone tissue through which the formed bone tissue can be vascularized. Growth factors such as vascular endothelial growth factor (VEGF) can be final delivered through these hollow fibers once the bone tissue has been formed to promote angiogenesis within the hollow structures following transplantation.

Delivery of nutrients and removal of waste products depends primarily on two factors: numbers of hollow fibers per unit volume of the culture chamber of the bioreactor and flow rates of nutrient solutions through the hollow fibers.

The hollow fibers of the present invention are those suitable for the delivery of nutrients and removal of waste in the bioreactor. The hollow fibers may be any shape, for example, they may be round and tubular or in the form of concentric rings. The hollow fibers may be made up of a resorbable or non-resorbable membrane. For example, suitable components of the hollow fibers include polydioxanone, polylactide, polyglactin, polyglycolic acid, polylactic acid, polyglycolic acid/trimethylene carbonate, cellulose, methylcellulose, cellulosic polymers, cellulose ester, regenerated cellulose, pluronic, collagen, elastin, and mixtures thereof. Moreover, the hollow fibers of the present invention include pores to allow the nutrients and waste to pass in and out of it. The pores of the hollow fibers are a sufficient diameter to allow the diffusion of a molecule from one side of the hollow fiber to the other side of the hollow fiber. Preferably, the molecules that may pass through the hollow fiber pores are about 0.002 to about 50 kDa, more preferably about 5-25 kDa, or most preferably 2-15 kDa.

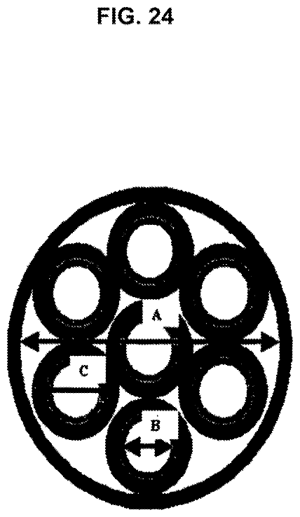

The number of hollow fibers per unit volume of the culture chamber of the bioreactor is determined based on the cross-section of the hollow fibers, the bioreactor per se, and the distance the bone-forming cells can live from the hollow fibers for nutrient delivery and waste removal. As an example of determining the number of hollow fibers per unit volume, FIG. 24 illustrates the cross section of a hollow fiber bioreactor. Assume the bioreactor cross section inner diameter (ID) is 2 cm (A), one hollow fiber ID is 1 mm (B), and the distance of cells can live from any conduit for nutrient delivery and waste removal is 20 μm, the ID of the circular area where nutrient deliver and waste remove by one hollow fiber (C) should equal to B+20×2 μm. Thus the number of hollow fibers needed for bioreactor can be calculated as follows:

Bioreactor ID(A)=2 cm 1)

Hollow Fiber ID(B)=1 mm 2)

Distance of Cells Can Live From Any Conduit for Nutrients Delivery and Waste Removal approximates 20˜30 μm depending on the diffusion rates of the nutrient molecules. According to human physiology, it is rare that any single functional cell of the body is more than 20-30 μm away from a capillary.

Calculation:Total Area of Cross-section of Bioreactor=(A/2)²*π=(2 cm/2)²*π=(10 mm)²*π=100 mm²*π 3)

Total Area of Cross-section of One Hollow Fiber=(B/2)²*π=(1 mm/2)²*π=0.25 mm²*π

Total Area of Nutrients Delivery and Waste Removal of One Hollow Fiber=(C/2)²*π=(1 mm/2+0.02 mm)²*π=(0.5 mm+0.02 mm)²*π=0.2704 mm²*π

Number of Hollow Fibers for Bioreactor with Cross-Section ID of 2 cm=100 mm²*π/0.2704 mm²*π=369.82 370

Percentage of Total Area Covered by Hollow Fibers=(0.25 mm²*π)*370/100 mm²*π*100=92.6% Percentage of Total Area Covered by Nutrients Delivery and Waste Removal=(0.2704 mm²*π)*370/100 mm²*π*100=100.48%

Although the flow of nutrient solutions through the hollow fibers will generate some minimal turbulent flow of solutions through the bulk volume of the growth chamber of the bioreactor, the primary mechanism for nutrient dispersal through the growth chamber and to the cells in culture will be diffusion and/or the alternating positive and negative pressure applications applied to the deformable bioreactor wall used to apply stress/strain to the demineralized bone, cells, and extracellular formed/forming matrix mixture during the culture process. Diffusion of nutrients from capillary beds in tissue typically limits the provision of nutrients (for example oxygen, glucose, etc.) to 20-30 μm from an individual capillary. Thus, if diffusion were the sole determinant of nutrient delivery and waste removal, it should be expected that cells located more than 20-30 μm from a hollow fiber will receive less nutrients and exist in a greater concentration of waste byproducts than cells close to a hollow fiber. With application of stress/strain to the demineralized bone, cells, and extracellular formed/forming matrix mixture via alternating applications of positive and negative pressure, it becomes possible to affect greater nutrient solution delivery and waste removal permitting cultivation of cells at greater distances from the hollow fibers than would be allowed by simple diffusion.

Shear stress to cells present in the bioreactor due to flow of nutrient solution will be minimal. Thus, optional addition of mechanical stress and strain to the forming bone matrix will occur primarily via manipulation of the inner vessel in the bioreactor used to contain the demineralized bone, cells, and extracellular formed/forming matrix. This component of the bioreactor includes the option of placing an inner vessel constructed of a deformable material within an outer vessel to which cyclic positive and negative pressure can be applied via a port in the outer vessel wall. It is to be expected that such positive and negative pressures will be minimal and designed to gently compress and expand the forming extracellular matrix in order to provide cyclic mechanical stimulation to the cells contained within the inner vessel of the bioreactor and to promote nutrient solution flow into, through, and out of the bioreactor containing the cells and matrix mixture.

In addition to the cyclic mechanical stimulation to cells contained within the inner vessel of the bioreactor, the inclusion of a series of micro-electrodes within the inner wall of the inner vessel in liquid contact with the forming, or formed, extracellular matrix will allow cyclic, low-level, electrical stimulation of cells and/or the creation of a small electrical gradient from one end to the other end, or side to side, of the bioreactor for use in electrical stimulation of cellular metabolism during induced new bone formation. This cyclic electrical stimulation can occur concurrent with, or not concurrent with, other mechanical or media changes to the forming, or formed, extracellular matrix containing the cells being manipulated to form new bone or bone-like tissue(s).

One aspect of the present invention is practiced by sterilizing all aspects of the bioreactor (tubing, fittings, valves, reagent (solution) containers, filters, sampling ports, bioreactor components, etc.).

The bioreactor 100 as shown in FIG. 21 illustrates an example of a hollow fiber bioreactor system of the present invention. The bioreactor 100 as set forth in FIG. 21 is aseptically assembled such that the hollow fibers 120 are connected to the inlet end-plate 106 and drawn through the tubular vessel 103 of the bioreactor 100 allowing the tubular vessel 103 of the bioreactor to be attached to the inlet end-plate 106 forming a water-tight seal. The non-connected end of the hollow-fibers 120 is then carefully attached to the outlet end-plate 102 forming a water-tight seal. Once the bioreactor is assembled, the ground demineralized bone can be rehydrated, if not already done so, and cells added via the injection ports 104 and 105. The bioreactor 100 is attached to at least one inlet port 107 and at least one outlet port 101 and the flow of nutrient solution from the nutrient reservoir 112 through the hollow fibers is initiated. The nutrients are delivered from the nutrient reservoir 112 through a noncytotoxic and nonhemolytic tubing 115 connected to the outlet port of nutrient reservoir 113 and the inlet port of the bioreactor 107. The flow is initiated and maintained in manners known in the art, but is preferably conducted centrifugal forces or a pump 114, such as a peristaltic pump, sufficient to cause the flow of media and waste products through the bioreactor 100. A pump 114 is preferably used to control the flow rate of the nutrients. Initiation of flow of nutrient solutions is important in that the cells contained in the bioreactor are labile to nutrient deprivation and thus the time between addition of cells to the bioreactor and initiation of nutrient solution flow should not exceed a time in which the specific cell population in the nutrient solution used to pack them becomes depleted of nutrients or changes pH to an extend that the cells become metabolically stressed. Additional reagents may added through a reagent addition port 109 as described above. Moreover, the waste generated from the bioreactor is removed through a tubing 108 connected to the outlet port 101 of the bioreactor 100 and the inlet port 110 of the nutrient reservoir 112. The eluent of medium from the bioreactor may be monitored to assess for proteins associated with bone formation, waste products, and nutritional capacity of the cells and demineralized bone, as described. The medium may also be recycled and recirculated into the nutrient reservoir 112 through a recycling inlet port 110. The nutrient solution in the nutrient reservoir 112 may be changed through the reagent addition port 109. One skilled in the art would appreciate when the nutrient solution should be changed. Preferably, the nutrient solution is changed at least once a week.

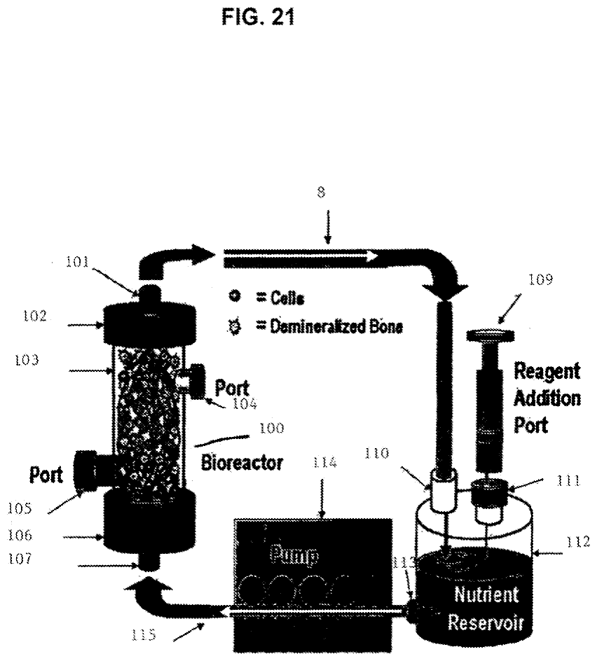

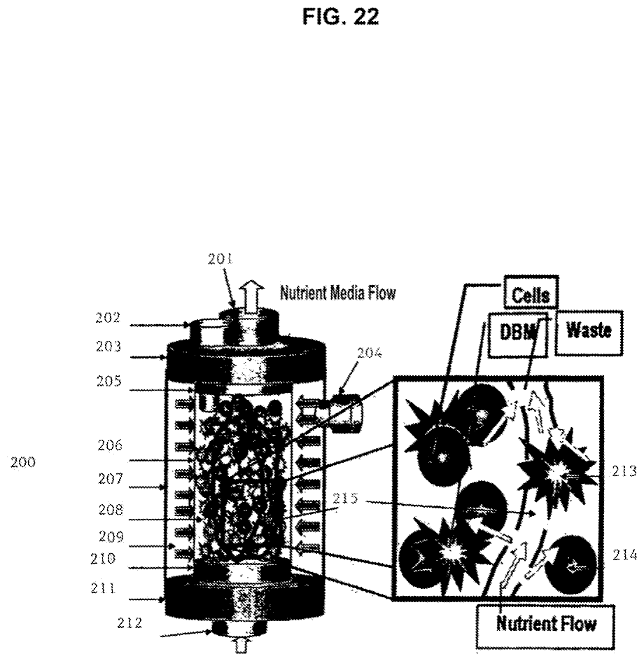

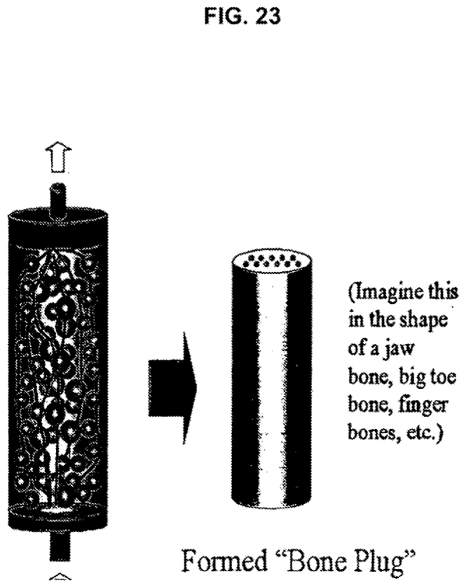

Referring to FIG. 22, the nutrient delivery and waste removal via hollow fibers of a bioreactor 200 of the present invention is depicted. The bioreactor 200 is aseptically assembled such that the hollow fibers 215 are connected to the inlet end-plate 210 and drawn through the tubular inner chamber 208 of the bioreactor allowing the tubular inner chamber 206 of the bioreactor 200 to be attached to the inlet end-plate 210 forming a water-tight seal. The ground demineralized bone is added into the inner-most volume of the inner vessel 206 before or following rehydration until it fills the inner-most volume. If the ground demineralized bone is rehydrated prior to or concurrent with the addition to the inner-most volume, it is mixed with the cells to be used at an appropriate seeding density, i.e. number of cells/unit volume of extra-particle space. If the ground demineralized bone is not rehydrated prior to addition to the inner-most volume, the bone will need to be rehydrated prior to addition of cells once the bioreactor is fully assembled. The non-connected end of the hollow-fibers 215 is then carefully attached to the outlet end-plate 205 forming a water-tight seal. This inner chamber 206 is now ready for insertion into the outer chamber 209 component of the bioreactor 200. This is accomplished by sliding the outer most diameter of one of the end-plates 211 through the internal lumen of the outer chamber 209 until the remaining end-plate 203 can form a water-tight seal with the inner diameter of the outer chamber 209. As an alternative method, the assembled inner chamber 206 can simply be inserted into the outer chamber 209 by guiding (pressing) the end-plates, 203 and 211, into the guide holes present in the inner faces of the outer chamber 209. Once the bioreactor is assembled, the ground demineralized bone can be rehydrated, if not already done so, and cells 214 may be added via the injection ports, 202 or 204. The flow of the nutrients would enter via at least one inlet port 212 and exit through at least one outlet port 201.

The deformable wall of the inner chamber of the bioreactor may be constructed out of a flexible permeable barrier and a fine deformable mesh that can be molded to a specific shape as needed. The flexible permeable barrier is mechanically supported by a fine mesh, which is present either on the inside or the outside of the flexible permeable barrier. The flexible permeable barrier is made of any suitable resorbable or non-resorbable membrane, such as those comprising polydioxanone, polylactide, polyglactin, polyglycolic acid, polylactic acid, polyglycolic acid/trimethylene carbonate, cellulose, methylcellulose, cellulosic polymers, cellulose ester, regenerated cellulose, pluronic, collagen, elastin, or mixtures thereof. The fine mesh is suitably made up of sterilizable materials, such as stainless steel, titanium, plastic polymer, nylon polymer, braided collagen, and silk polymer, but must be capable of deforming to any desired shape. The fine mesh may have any suitable pore size dictated by the desired bone plug properties. For example, suitable pore sizes for the mesh is between about 0.1 to 10 mm and, preferably, 1-5 mm. The deformable wall may be made to be permeable for some metabolites and not others. For example, the deformable wall may be made to not be permeable to small or large molecular weight metabolites. In particular, a small molecular weight metabolite would fall within the range of 0.001-25 kDa, preferably 0.1-2.5 kDa. A larger molecular weight metabolite would fall within the range of 25-200 kDa, preferably 25-50 kDa. The deformable wall may further be constructed to allow for its use in the bioreactor of the present invention. For example, the tensile properties of the deformable wall should make it capable of deforming under the cyclic negative and positive pressure, such as between 10-30 mmHg. The mesh used to construct the deformable wall preferably will conduct an electrical current. The resorbable or non-resorbable hollow fibers can be used to deliver nutrients and remove waste for the inner chamber. The deformable inner chamber can be contained within a nondeformable outer chamber. The cyclic application of positive and negative pressures to the deformable wall of the inner chamber of the bioreactor to be used in the in vitro growth of bone or bone-like tissue serve to transform this bone or bone-like tissue into bone following transplantation into a recipient.

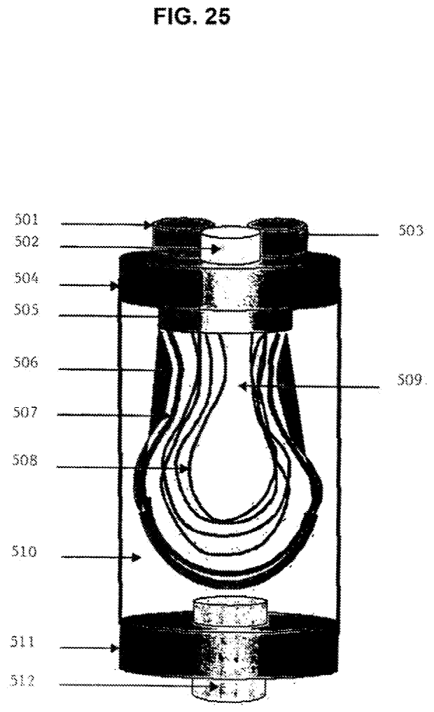

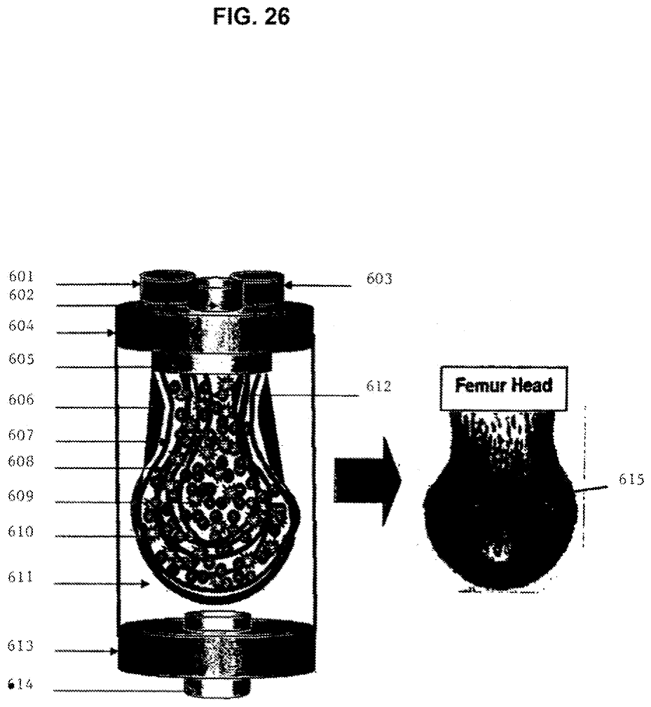

Inlet and outlet ports of the outer chamber can deliver nutrients and remove waste for this deformable chamber (FIGS. 25 and 26). For example, FIG. 25 illustrates a hollow fiber bioreactor with an inner deformable chamber 509, wherein the deformable wall is comprised of a flexible permeable barrier 507 and a fine mesh 506. The bioreactor may contain one outer chamber 510 and one inner deformable chamber 509. The outer chamber 510 is closed by two end-plates 504 and 511 by means suitable for closing the chamber, such as an annular groove. The inner deformable chamber 509 is closed by one end-plate 505. The flexible permeable barrier 507 (non-resorbable membrane or resorbable membrane) and a fine mesh 506 are sealed to the plate 505 of the inner deformable chamber 509. The inner deformable chamber 509 can be deformed to the desired shape using a deformable metal mesh 506. At least one inlet 512 and at least one outlet 502 port is connected to the outer chamber 510 and are used for the nutrient delivery and waste removal in the outer chamber 510. Nutrient delivery and waste removal in the inner chamber 509 employ the use hollow fibers 508 connected to the at least one inlet 501 and at least one outlet 503 port on the outer chamber cover 504 and inner chamber cover 505.

FIG. 26 illustrates a femoral head formation using a hollow fiber bioreactor 600 of the present invention. The fine mesh 606 is deformed to the shape of a femoral head 615. A permeable membrane 607 is lined inside of the fine mesh 606. A mixture of demineralized bone materials 609 and cells 610 is added into the inner chamber 612 along with the hollow fibers 608 dispersed in the mixture of demineralized bone materials 609 and cells 610. The ends of hollow fibers 608 are connected to the two ports, inlet 601 and outlet 603, for nutrient delivery and waste removal from the inner chamber 612. The permeable membrane 607 and fine mesh 606 are sealed to the end-plate 605 of the inner chamber 612. The end-plate 605 of the inner chamber 612 is connected to the end-plate 604 of the outer chamber 611 through an annular groove: The nutrient is delivered into the out chamber through the inlet port 614 and the waste is removed from the outer chamber 611 through the outlet port 602.

The nutrient medium provided and the flow rate of this nutrient medium will vary depending on cell type added to the bioreactor, the packing density of the demineralized bone, presence/absence of a pre-added “extracellular matrix”, and numbers and kinds of hollow fibers contained within the inner vessel of the bioreactor. Nutrient flow will continue until such time as it has been previously determined that the appropriate matrix (structure) has been obtained. At this time, the bioreactor is aseptically dismantled and the bone or bone-like structure aseptically removed for further use.

The formed new bone can consist of a nonmineralized and nonload-bearing osteoid-like material that will mineralize when transplanted into a heterotopic or orthotopic site in a patient or a partially mineralized and partially load-bearing osteoid material that will further mineralize when transplanted into a patient. Given time, it is also possible to produce an almost completely mineralized bone-like tissue that will be load-bearing when implanted clinically.

In another aspect of the invention, the demineralized ground bone and bone-forming cells may form an extracellular matrix that is capable of forming bone when implanted in a patient. In this manner, the demineralized bone and cells may be gelled in a viscous material and have non-loading bearing implantable material that will form in vivo similar to the in vitro bone-forming process described above.

The bone, bone-like tissue, and extracellular matrix made according to the present invention is suitable for transplantation into a patient in need thereof. As one having ordinary skill in the art would appreciate, the bone, bone-like material or tissue, and extracellular matrix can be made into a desired shape that the body will remodel into the appropriate bone when implanted into a patient in some clinical application. For example, as shown in FIGS. 27A and 27B, bone plugs formed in bioreactors of the present invention can have varying shapes and sizes. In particular, the bone plugs depicted in FIGS. 27A and 27B were generated after 4 weeks of incubation of ground demineralized bone particles and human fibroblasts in the bioreactor.

Moreover, the bone, bone-like tissue, and extracellular matrix may be further treated prior to implantation in manners known in the art. For example, these materials may be acellularized using known methods prior to implantation. Preferred methods of acellularization include, but are not limited to, methods described in U.S. patent application Ser. Nos. 09/528,371 and 09/660,422, which are hereby incorporated in their entirety. The acellularized bone, bone-like tissue and extracellular matrix is within the scope of the present invention. In addition, these acellularized may be recellularized by known methods either in vitro or in vivo. Alternatively, any residual resorbable hollow fibers present in the bone, bone-like tissue, or extracellular matrix may be removed using hydrolytic enzymes, such as cellulase, chitinase, collagenase, elastase, proteases such as chymotrypsis, trypsin, ficin, papain and/or specific enzymes that are capable of degrading the polymers comprising the resorbable and non-resorbable hollow fibers and dialysis films. Other known methods of processing bone prior to implantation are further within the scope of the present invention.

### Example 1

The following examples are for purposes of illustration only and are not intended to limit the scope of the appended claims.

Diaphysyl shafts (total of approximate 520 grams wet weight of bone material) from the long bones and ribs of a given donor (human donor information is confidential) were mechanically debrided (as disclosed in co-pending U.S. patent application Ser. No. 10/108,104, incorporated by reference herein) to remove associated periosteal tissue and bone marrow in the intramedulary canal. The shafts and ribs were then cut into linear pieces with widths, thickness, and lengths approximating <45 mm x<45 mm x<6 cm using a bone saw. A cut piece of cortical bone (wet weight 48 grams) was then loaded individually into the load chute of the cutting device and the clamping cylinder was locked into the closed position. The cutting slide having the cutting blade disposed therein was activated and cut fiber bone was collected into the receiving bin. A total of 42 grams of fiber bone were accumulated during the 60 cutting cycles (cutting cycle equals one back/forthpass of the cutter/cutter slide across the bone surface) for approximately 70 seconds with additional bone materials being added to the feeder chute at each cutting event. After each cutting event, another cortical shaft and/or cortical pieces were added and another cutting event was initiated. The amount of the bone materials loaded into the chute for each cutting event varied. However, the number of cutting events performed were sufficient to accumulate a bulk fiber mass of approximately 490 grams (wet weight).

The cut fiber bone was stored in a sterile container in the freezer (minus 80 C.) for three days. Prior to demineralization, the cut fiber bone was cleaned with LifeNet's patented ALLOWASH technology. For demineralization, a total of 463 grams of bone materials were added to the Pulsatile Acid Demineralization (PAD) chamber (as disclosed in co-pending U.S. patent application Ser. No. 09/655,371 herein incorporated by reference) and demineralized to 2.5% residual calcium using 2 cycles of 0.5 N HCl and acid volumes of 4.0 liters/cycle and 3.0 liters/cycle, 1 cycle of ultrapure water of 3.0 liters/cycle, and 2 cycles ultrapure water plus buffer of 3.0 liters/cycle to terminate the demineralization process. The bone fibers were finally washed in 3.0 liters of ultrapure water and stored frozen at minus 80 C in a sterile container.

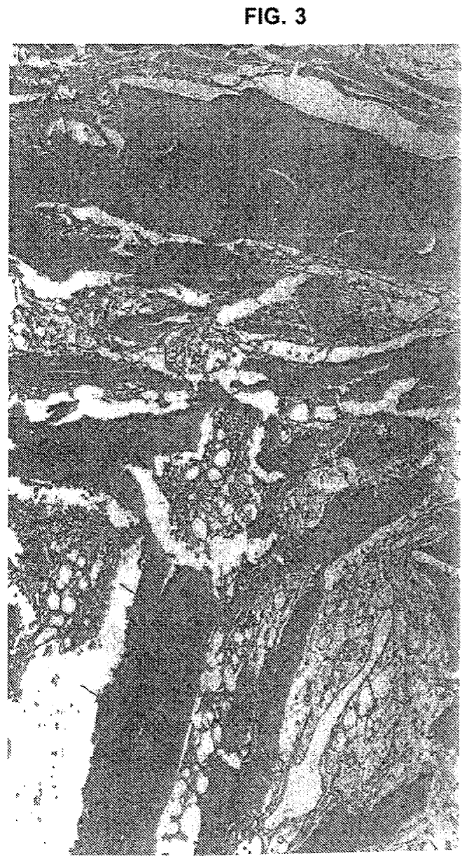

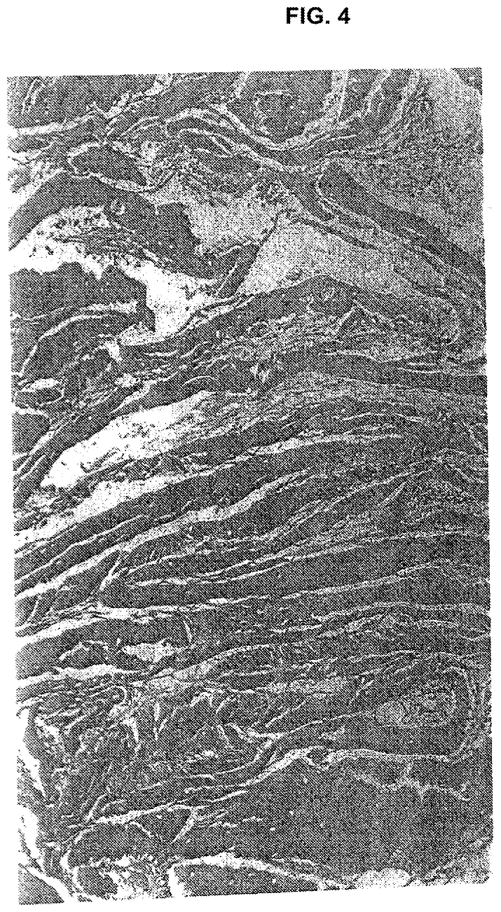

Aliquots of the demineralized fiber bone were removed from a sterile container and transferred to the animal implantation laboratory. Aliquots of fiber bone (20 and 40 mg wet weight) were manually compacted and implanted intramuscularly into the hindquarters of athymic (nude) mice as compressed fiber bone materials using established Institutional Animal Care and Use Committee approved protocols (Old Dominion University). After 28 days of implantation, the implanted materials were explanted and the explants fixed in formaldehyde. The fixed explants were embedded in paraffin and sectioned for use in preparation of histology slides. The prepared histology slides were stained using Hematoxylin and Eosin (H&E staining) and viewed under the microscope for induced new bone formation. The induced new bone formation is illustrated in FIG. 3. Induced new bone formation was determined using histomorphometry and the bone materials were determined to have induced significantly more new bone than non-osteoinductive controls, i.e. the fiber bone was deemed to be osteoinductive using the nude mouse bioassay model.

### Example 2

### In Vitro Attachment of Fibroblast Cells to Fiber Bone

The attachment of fibroblast cells to fiber bone may be quantitated using the methyltetrazolium dye assay method (MTT) where metabolic activity reduces the methyltetrazolium dye to an insoluble (chromogenic) substrate that can be quantitated using the spectrophotometer. In this particular assessment, cell attachment is compared with cell attachment to particle bone (cortical bone ground, using impact fragmentation) ground to a particle size range of 250 to 710 microns, demineralized and used in equal gram equivalents.

Fibroblast cells (NIH 3T3) were chosen for the study in that these cells represent relatively undifferentiated cells present in the body and are presumed to represent those cells that primarily migrate to the site of implantation of demineralized bone such as used in nude (athymic) mouse implant studies to assess the osteoinductivity of demineralized bone.

Fibroblast cells (1-5×10⁵ cells/ml) grown in RPMI 1640 tissue culture medium (supplemented with 10%, by volume, fetal calf serum (FCS) and glutamine) were harvested from the T-75 culture flasks using trypsinization. The residual trypsin associated with the cells put into suspension was neutralized by resuspending the cells in fresh RPMI 1640 tissue culture medium (supplemented with 10% FCS). Demineralized fiber bone (100 mg, wet weight) was aliquoted into replicate (20) 15 ml sterile centrifuge tubes and demineralized particle bone (100 mg, wet weight) was aliquoted into replicate (20) 15 ml sterile centrifuge tubes. The twenty tubes of fiber bone and 20 tubes of particle bone were divided into two groups each of 10 replicates such that one group of 10 would be incubated with tissue culture medium without cells and the remaining group of 10 would be incubated with tissue culture medium with cells. Each tube received 5 mls of medium (medium containing or not containing cells) such that tubes receiving medium with cells received approximately 5×10⁵ to 1×10⁶ cells/100 mg of demineralized bone (fiber or particle). The tubes were statically incubated at 37 C for one (1) hour, at which time the medium was decanted off of the bone and fresh medium (5 ml) added and decanted to affect a “washing” of the demineralized bone. This “washing” process was repeated a total of three times. All steps were conducted using aseptic techniques such that the demineralized bone could be incubated overnight at 37 C to permit the attached cells to proliferate.

Following the overnight incubation, the demineralized bone/medium/“cells” (if added in the centrifuge tube) were vigorously vortexed to dislodge cells and the medium decanted to a fresh centrifuge tube. The dislodged cells were concentrated by low speed (1,500 to 2,000 rpm in a clinical table top centrifuge) centrifugation and the medium decanted. The cell pellets were assayed using the standard MTT assay and the numbers of cells “quantitated” by comparison to a standard curve where known numbers of cells were aliquoted into centrifuge tubes, centrifuged to concentration and assayed.

Background absorbance values were obtained using the demineralized bone (fiber and particle) incubated in the absence of cells. On average, the fiber bone presented 1-5×10³ cells/100 mg of bone whereas the particle bone presented approximately 2-4×10² cells/100 mg of bone, i.e., an approximate 10-fold greater numbers of cells per unit wet weight of fiber bone to particle bone.

### Example 3

### In Vivo Attachment of Cells to Fiber Bone

Implantation of biomaterials into muscle pouches of athymic (nude) mice (two implants/mouse, implanted bilaterally in the gluteal region of the mouse) represents the current “gold-standard’ method of assessing the osteoinductivity of such biomaterials. Between 10 and 20 mg (dry weight) of biomaterials (demineralized bone in this example) are rehydrated with isotonic saline and implanted just under the fascia using a dental amalgum tool (such as typically used by a dentist to add the filing materials to a cavity formed in teeth).

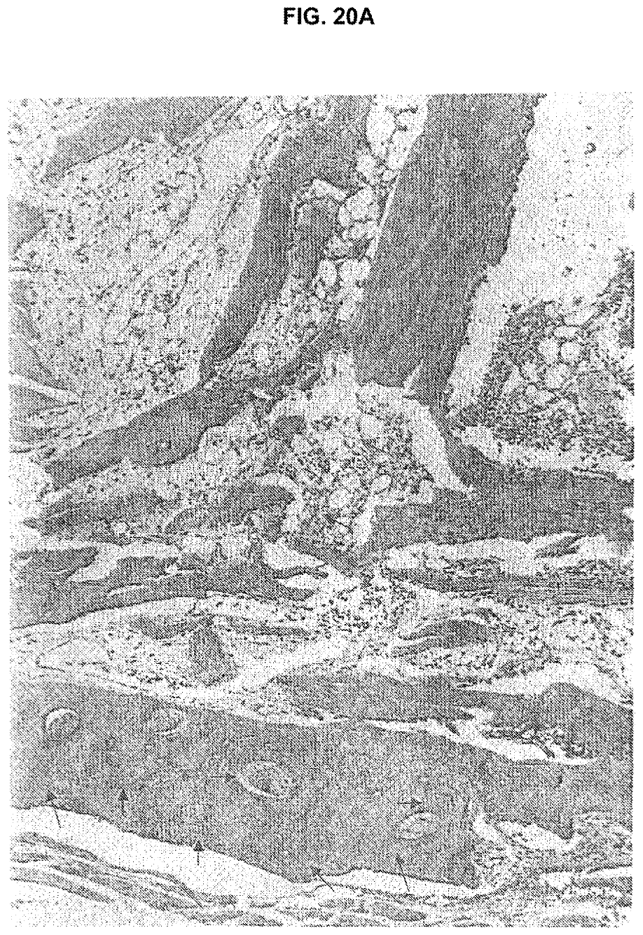

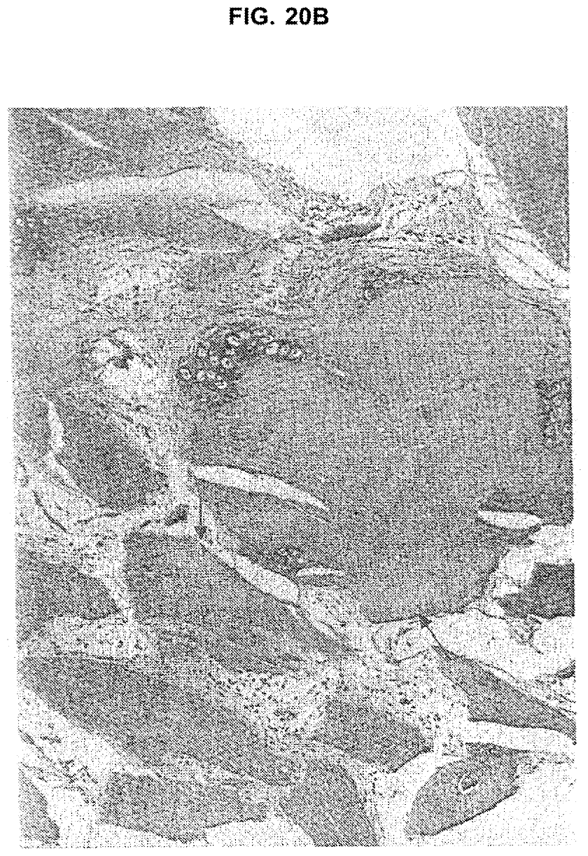

In this study, human “shaved” (fiber) bone and human “DMB Positive Control” (particle) bone were implanted into muscle pouches of athymic mice (two implants/mouse and three mice per implant group). The implanted materials were explanted after 28 days, and the explants (explanted as “hard” nodules) were fixed in buffered formalin. The samples were decalcified and embedded in paraffin prior to preparation of histological sections for staining (hematoxalin/eosin; H&E). As illustrated in FIGS. 20A (bone fibers) and 20B (control), both demineralized bone materials were osteoinductive, in that new bone formation was clearly visible in the histology sections. However, cells are more clearly visible along the edges of the fiber bone materials as shown in FIG. 20A as compared to the comparable edges of the particle bone materials as shown in FIG. 20B suggesting that cells migrating to the implant sites were more likely to bind tightly to the fiber bone than to the particle bone (although both demineralized bone biomaterials induced cells infiltrating the implant materials to differentiate into “osteoblast” or “osteoblast-like” cells and synthesize new bone matrix that stained comparably to implant bone).

### Example 4

### Growth of New Bone Using a Sample Inner Vessel Consisting of Dialysis Membrane Tubing in a Circulating Solution of Nutrient Solution

Dialysis tubes (Spectrum, Spectra/Por) made with different membrane pore sizes (MWCO 10,000-25,000) and different material (regenerated cellulose or cellulose ester) were used for musculoskeletal bone tissue regeneration. The hydrogen peroxide in sterile dialysis tubes was removed and the tubes were soaked in tissue culture media for 1-2 hours in order to remove all remnants of hydrogen peroxide. Demineralized bone matrices were weighed aseptically and hydrated with cell suspension (human dermal fibroblasts or human periosteal cells) in RPMI 1640 tissue culture medium. The DBM and cell mixtures were introduced into dialysis tubes and the tubes were incubated in culture media containing 2% FBS, 50 μg/ml L-ascorbic acid, 1 μM dexamethasone, and 50 mM beta-glycerolphosphate. The dialysis system was incubated either under static (that means the dialysis tubes are incubated in a media container), stirred dynamic (that means the dialysis tubes are incubated in a media container which stays on stir plate to give constant mixing speed), or fluid-flow dynamic (that means the dialysis tubes are incubated in a media flow chamber which controls the media flow rate for dialysis tubes by peristaltic pump) conditions. The culture media were replaced by fresh media once a week to keep sufficient nutrients for cell growth and differentiation.

During various time of incubation (1-7 weeks), the culture media were taken out from the containers for osteocalcin quantitation by ELISA, the tissues from the dialysis tubes were taken out for histology analysis, alkaline phosphatase quantitation, percentage of calcium quantitation, and double strand DNA quantitation. The samples of culture media were taken out from bioreactor each week for osteocalcin quantification by ELISA. FIG. 30 shows the time course of the bone protein, osteocalcin, levels for different cell seeding densities and identifies that the osteocalcin levels in the culture media increased significantly for the first 4 weeks and were more consistent after fourth week. Similarly, the osteocalcin levels normalized by the amount of DNA in the bone plugs generated in the bioreactor was also calculated based on the various cell seeding densities and incubation time. As shown in FIG. 30, the lowest seeding density (0.5 million fibroblast cells per 100 mg of DBM) showed the highest osteocalcin level from second to fifth week of incubation.

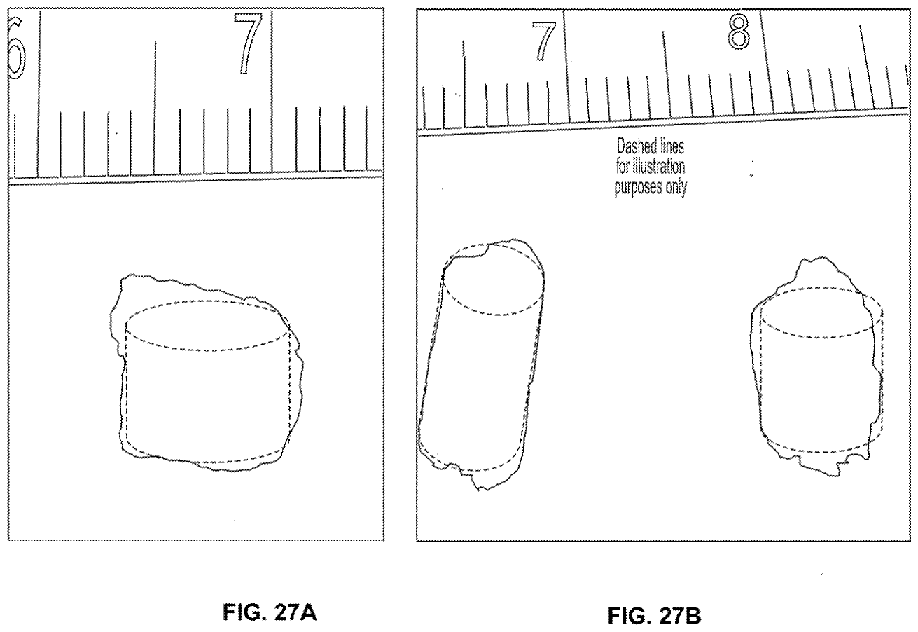

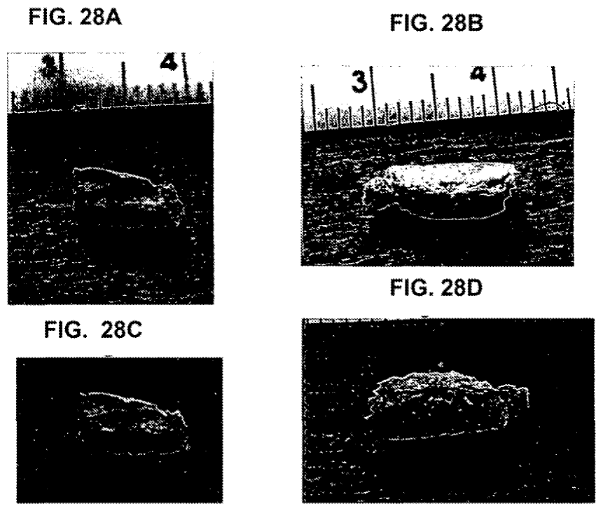

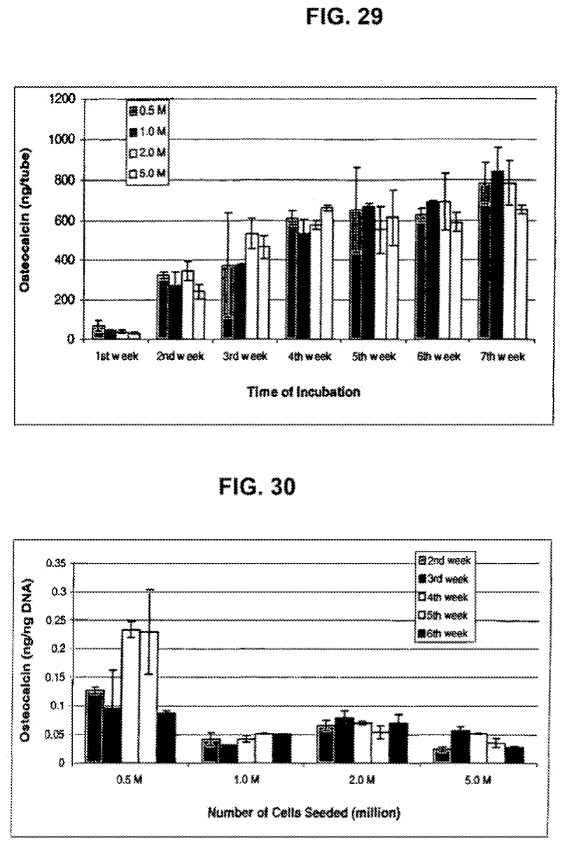

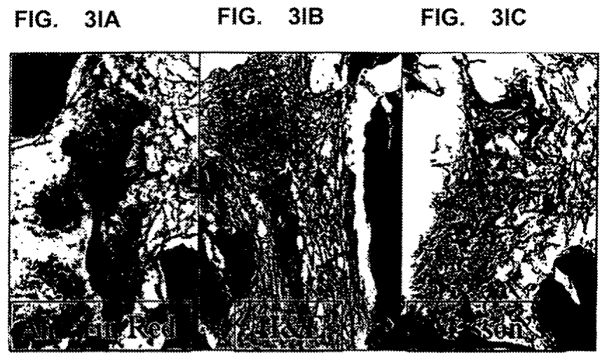

Various bone plugs produced according to this example were further examined. Specifically, some of the bone plugs formed according to this example are depicted in FIGS. 27A and 27B, which indicates the various shapes and sizes available to the person performing the invention. Additionally, FIGS. 28A-28D illustrate the bone plugs generated in the bioreactor that are subsequently freeze-dried. The shapes of these bone plugs reflect the shape of the deformable inner vessel of the bioreactor. FIGS. 31A-31C and 32A-32C illustrate the histological analysis of a bone plug generated in a bioreactor at 200× and 400× magnification, respectively. The “bone plug” generated in bioreactor was embedded and sectioned and the sections were stained with the Alizarin Red, H&E, and Masson's Trichrome methods. The Alizarin Red staining revealed the calcium deposition in newly formed extracellular matrix. H&E staining revealed the changes in fibroblast morphology and new extra-cellular matrix (ECM) production that appeared to be “osteoid” formation. Masson's Trichrome staining suggested that the newly formed extracellular matrix contained significant quantities of collagen.

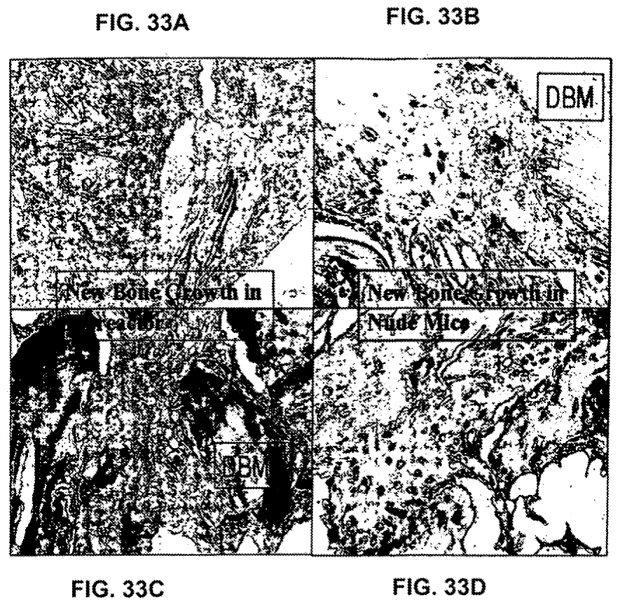

FIGS. 33A-33D illustrate the H&E staining of a bone plug generated in a bioreactor and an analogous bone plug generated from heterotopic implantation of DBM in a nude mouse (400× magnification). The new bone growth in a bioreactor (FIGS. 33A and 33B) of the present invention after 4 weeks incubation was compared to the new bone growth in a nude mouse (FIGS. 33C and 33D) 4 weeks after DBM implantation. The changes in fibroblast morphology and new extracellular matrix production appeared on both samples.

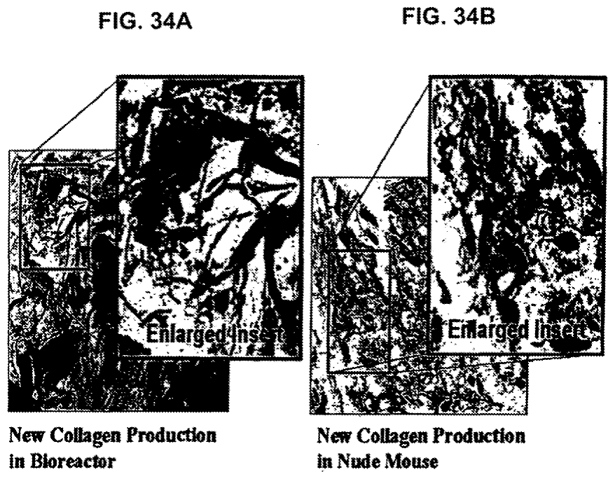

FIGS. 34A-34B illustrate the Mason's Trichrome staining of a bone plug generated in a bioreactor (FIG. 34A) and an analogous bone plug generated from heterotopic implantation of DBM in a nude mouse (FIG. 34B) (400× magnification). Significant amounts of new extracellular matrix were produced around cells and stained as collagen fibril for both “bone plug” generated in a bioreactor and explants from a nude mouse.

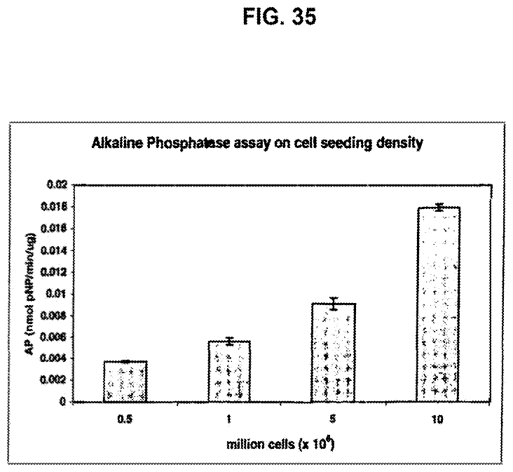

FIG. 35 illustrates the alkaline phosphatase activity for bone plugs generated in a hollow fiber bioreactor with various cell seeding densities. The group at a cell seeding density of 1×10⁷ human periosteal cells per 500 mg of DBM showed significantly higher alkaline phosphatase activity than other groups tested.

### Example 5

### Growth of New Bone Using a Prototypic Hollow-Fiber Containing Bioreactor

The bioreactor was constructed from glass tubing (inner diameter, 5 mm; length, 50 mm) and contained forty porous regenerated cellulose hollow fibers (outer diameter, 216 μm; inner diameter, 200 μm; MWCO of 18,000; Spectra/Por®; Spectrum Laboratories, Inc.; Laguna Hill, Calif.). The hollow fibers were embedded in biomedical grade silicon rubber (Nusil Silicone Technology, Carpenteria, Calif.).

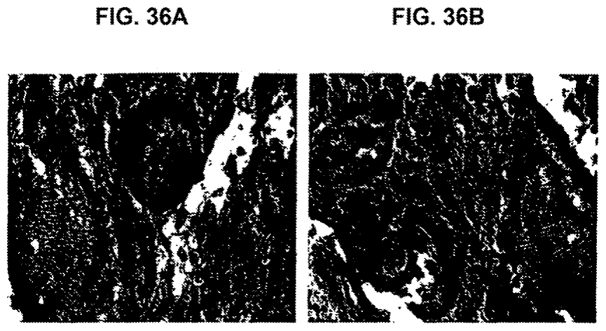

To determine the optimal cell seeding density in the bioreactor system, human periosteal (HPO) cells were inoculated into the bioreactor at various cell density of 0.5×10⁶, 1×10⁶, 5×10⁶, and 1×10⁷ cells with DBM (1.5 cc or 500 mg). The culture medium used comprises Dulbecco's modified Eagle's medium (DMEM) supplemented with antibiotics, ascorbic acid, beta-glycerophosphate, dexamethasone, and 2% fetal bovine serum (FBS). Two hundred and fifty ml of cell culture medium was recirculated with a medium flow rate of approximately 5 ml/min. After inoculation, the bioreactors were perfused using a peristaltic pump and maintained in a 5% CO₂/95% air incubator. After 5 days, the samples of cells with DBM were removed and in vitro alkaline phosphatase assay was performed. FIGS. 36A-36B represent the various cell seeding densities of HPO cells and the activities of alkaline phosphatase from the in vitro alkaline phosphatase assay. These data demonstrate that HPO cells at a density of 1×10⁷ cells have significantly higher alkaline phosphatase activities than other groups with different cell seeding densities tested.

To study the growth of new bone or bone-like tissue using hollow-fiber bioreactor system, the bioreactor was inoculated with 1×10⁷ cells and DBM (1.5 cc or 500 mg) through either end into the extracapillary space of the bioreactor. Dulbecco's modified Eagle's medium (DMEM) supplemented with antibiotics, ascorbic acid, beta-glycerophosphate, dexamethasone, and 2% fetal bovine serum (FBS) was used as culture medium throughout the experiments. Culture medium was changed weekly. Two hundred and fifty ml of cell culture medium was recirculated with a medium flow rate of approximately 5 ml/min. Diffusive nutrient supply and removal of metabolic waste products across the membrane of hollow fiber was advanced by constantly recirculating culture medium through the system using a peristaltic pump maintained in a 5% CO₂ incubator. After 3 weeks, samples were taken from the bioreactors, fixed in neutral buffered formalin, embedded in paraffin and sectioned. Sections were stained with Haematoxylin & Eosin. The results were illustrated in FIGS. 36A-36B showing H&E stained large cuboidal-shaped cells with deposition of collagen and organic bone matrix at 400× magnification.

Each of the patents and publications cited herein are incorporated by reference herein in their entirety. It will be apparent to one skilled in the art that various modifications can be made to the invention without departing from the spirit or scope of the appended claims.

While the invention has been described in connection with specific embodiments thereof, it will be understood that it is capable of further modifications and this application is intended to cover any variations, uses, or adaptations of the invention following, in general the principles of the invention and including such departures from the present disclosure as within known or customary practice within the art to which the invention pertains and as may be applied to the essential features hereinbefore set forth as follows in the scope of the appended claims. Any references including patents and published patent applications cited herein are incorporated herein in their entirety.

What is claimed:

1. An allogeneic bone material composition for implantation in a human patient, comprising:
(a) human demineralized cortical bone,
(b) human non-demineralized bone comprising cortical bone, cancellous bone, or a combination thereof,
(c) cells selected from the group consisting of osteocytes, pre-osteoblasts, osteoblasts and combinations thereof, and
(d) a biological fluid,
wherein the allogeneic bone material composition is stored frozen in a sterile container.

2. The allogeneic bone material composition of claim 1, wherein the composition comprises osteocytes.

3. The allogeneic bone material composition of claim 1, wherein the composition comprises osteoblasts.

4. The allogeneic bone material composition of claim 1, wherein the composition further comprises cells selected from the group consisting of stem cells, connective tissue progenitor cells, and combinations thereof.

5. The allogeneic bone material composition of claim 1, wherein the composition further comprises stem cells.

6. The allogeneic bone material composition of claim 1, wherein the osteocytes, pre-osteoblasts, or osteoblasts are suitable for forming an osteoid.

7. The allogeneic bone material composition of claim 1, wherein the biological fluid is a human biological fluid selected from the group consisting of plasma, bone marrow, blood, a human blood product and a combination thereof.

8. The allogeneic bone material composition of claim 1, wherein the demineralized cortical bone is in the form of particles.

9. The allogeneic bone material composition of claim 1, wherein the demineralized cortical bone is in the form of fibers.

10. The allogeneic bone material composition of claim 1, wherein the demineralized cortical bone contains calcium at a level of from 0.1 wt % to 7.7 wt %.

11. The allogeneic bone material composition of claim 1, wherein the demineralized cortical bone contains calcium at a level of from 1 wt % to 4 wt %.

12. The allogeneic bone material composition of claim 9, wherein the bone fibers have a length and thickness, and wherein the fiber length is greater than 10 to 200 times the fiber thickness.

13. The allogeneic bone material composition of claim 9, wherein the bone fibers have a length and thickness, and wherein the fiber length is greater than 40 to 100 times the fiber thickness.

14. The allogeneic bone material composition of claim 9, wherein the bone fibers have a length, and wherein the average fiber length is from 1 mm to 100 mm.

15. The allogeneic bone material composition of claim 9, wherein the bone fibers have a length, and wherein the average fiber length is from 20 mm to 30 mm.

16. The allogeneic bone material composition of claim 1, wherein the non-demineralized bone comprises cortical bone.

17. The allogeneic bone material composition of claim 1, the non-demineralized bone comprises cancellous bone.

18. The allogeneic bone material composition of claim 1, the non-demineralized bone is in the form of chunks.

19. The allogeneic bone material composition of claim 1, the non-demineralized bone is in the form of particles.

20. The allogeneic bone material composition of claim 1, further comprising a bone growth agent.

21. The allogeneic bone material composition of claim 20, wherein the bone growth agent is selected from the group consisting of bone morphogenic proteins, angiogenic factors, growth and differentiation factors, mitogenic factors, osteogenic factors, chondrogenic factors and combinations thereof.
